SEQUENCE LISTING (113 sequences)

SEQ ID NO: 1 - protein, 657 aa, Fusarium graminearum
MGHHHHHHSSGHIEGRHMASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 2 - protein, 639 aa, Fusarium graminearum
ASAPIGSAIPRNNWAVTCDSAQSGNECNKAIDGNKDTFWHTFYGANGDPKPPHTYTIDMKTTQNVNGLSVLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFEGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSFSGGVFEKNGEVYSPSSKTWTSLPNAKVNPMLTADKQGLYKSDNHAWLFGWKKGSVFQAGPSTAMNWYYTSGSGDVKSAGKRQSNRGVAPDAMSGNAVMYDAVKGKILTFGGSPDYTDSDATTNAHIITLGEPGTSPNTVFASNGLYFARTFHTSVVLPDGSTFITGGQRRGIPFEDSTPVFTPEIYVPEQDTFYKQNPNSIVRAYHSISLLLPDGRVFNGGGGLCGDCTTNHFDAQIFTPNYLYDSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 3 - protein, 496 aa, Colletotrichum spinosum
MGQGVGQWGPLVKFPIVPVAVALIPESGNMLVWSSGWPNRWTNAGNGKTFTSLYDVSTGKAGDAIVQNTQHDMFCPGTSLDVEGRIIVTGGSSAAKTSVLDFRKGESSSWAPLSNMQISRGYQSSCTTSEGKIFVIGGSFSGAGTRNGEIYDTASNKWTKLAGCPVKPLVMQRGLFQDSHTWLWSWKNGSVLQAGPAKQMNWYDTKGTGANTPAGLRGADDDAMCGVSVMFDAVAGKIFTYGGGKAYTGYAASSNAHILTLGEPGQQVQVQKLANGQYNRGFSNAVVLPDGRIWVVGGMRQMQLFSDNTPQLTPELFDPATGKFTPTAPHAIPRNYHSTALLMADATVWSGGGGLCGANCKENKFDGQFWSPPYLFEADGKTPAQRPVIDSLSDKTVRAGAPLVVTMKDEGKYTFSMLRVSATTHTVNTDQRRIPLDGQDGGDGKSFTVNMPGDYGVVIPGYYMMFAMNEAGVPCVAKFFKVALGQTGLEHHHHHH

SEQ ID NO: 4 - protein, 483 aa, Colletotrichum spinosum
GQGVGQWGPLVKFPIVPVAVALIPESGNMLVWSSGWPNRWTNAGNGKTFTSLYDVSTGKAGDAIVQNTQHDMFCPGTSLDVEGRIIVTGGSSAAKTSVLDFRKGESSSWAPLSNMQISRGYQSSCTTSEGKIFVIGGSFSGAGTRNGEIYDTASNKWTKLAGCPVKPLVMQRGLFQDSHTWLWSWKNGSVLQAGPAKQMNWYDTKGTGANTPAGLRGADDDAMCGVSVMFDAVAGKIFTYGGGKAYTGYAASSNAHILTLGEPGQQVQVQKLANGQYNRGFSNAVVLPDGRIWVVGGMRQMQLFSDNTPQLTPELFDPATGKFTPTAPHAIPRNYHSTALLMADATVWSGGGGLCGANCKENKFDGQFWSPPYLFEADGKTPAQRPVIDSLSDKTVRAGAPLVVTMKDEGKYTFSMLRVSATTHTVNTDQRRIPLDGQDGGDGKSFTVNMPGDYGVVIPGYYMMFAMNEAGVPCVAKFFKVAL

SEQ ID NO: 5 - protein, 728 aa, Hypomyces rosellus
MADQQTVLSVSVPGYIRLEDISYSSSASKTFIILSSHSLLIIISLYVNMKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 6 - protein, 680 aa, Fusarium graminearum
MKHLLTLALCFSSINAVAVTVPHKAVGTGIPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 7 - protein, 685 aa, Purpureocillium lilacinum
MKLLGTVAALALCDAASHTYAVAVKPRPHTAMPNLAARGTEMSLMAAKPIGNAINRAGWKVTCDGEEQGNECAKAIDGDNNTMWHTAWQNDNPPPPHTITVDMGSAQTINGISVLPRQDGSEHGWIARHDVLVSNDGQTWGDPVATGTWYTDATAKYANFEPRSARYVRLVARSEAQGRPWTSIAELNVYRADGPPVPKNGIGKWGLTLDFPVVPVAGVVDPLTGKVVVWSAYENDQYEGSPGGWTLTSTWDPATGEVTERNVTNIGHDMFCPGVSLDASGRVVVTGGSNAQKTSFYDAATEAWVPGPDMKTPRGYQASATCSDGRVFTIGGSWSGGQFEKNGEVWDPKTNSWRALPGAAVKPMLTKDRGGIYRADNHAWLFGWRNGSVFQAGPSTAMNWYYTAGDGRVRSAGQRRAPRGADPDAMCGNAVMFDATAGKILTVGGAPHYEDADATTNAHVLTLGDAGAAPKVVFAGNGMAHPRIFANAVVLPDGTVFVTGGQQHAELFKDTTPQLTPELYDPALGAFVEQAPNSVVRVYHSMALLLPDATVLSGGGGLCGGACDTNHFDAQVFSPRYLFDGEGQPAARPKIRAVASKEVHAGDAIKVTTDGPVKSAALVRYGSATHSVNTDQRRVPLTLRQDGGSYAYSADLPRDLGVLLPGYWMLFVMNDKGVPSVAATVKVLL

SEQ ID NO: 8 - protein, 680 aa, Fusarium graminearum PH-1
MKHFLSLALCFSSINAVAVTVPHKSGGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSITKASLIRYGTATHTVNTDQRRIPLTLTNNGGNSYSFQVPSDSGVALPGYWMLFVMNSAGVPSVASTIRVTQ

SEQ ID NO: 9 - protein, 680 aa, Fusarium pseudograminearum CS3096
MKHFLSLALCFSSINAVAVTVPHKVAGTGNPEGSLQFLSLRASAPIGSAISRNNWAVTCDSAQSGNECNKAIDGSKDTFWHTFYGANGDPKPPHTYTIDMKTTQNVNGLSMLPRQDGSQNGWIGRHEVYLSSDGTNWGSPVASGSWFADSTTKYSNFETRPARYVRLVAVTEANGQPWTSIAEINVFQASSYTAPQPGLGRWGPTIDLPIVPAAAAIEPTSGRVLMWSSYRNDAFGGSPGGVTLTSSWDPSSGVVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADRQGLYRSDNHAWLFGWKKGSVFQAGPSTAMNWYYTSGNGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSAQSVKVGGRITMSTDSSITKASLIRYGTATHTVNTDQRRIPLTLTNNGGNSYSFQVPSDSGVALPGYWMLFVINSAGVPSVASTIRVTQ

SEQ ID NO: 10 - protein, 639 aa, Hypomyces rosellus
ASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 11 - protein, 680 aa, Fusarium sporotrichioides
MKHLLTLALCFSSINAVAITNPHKTAGHDHPEGSLQFLSLRASAPIGSAISRNNWAVTCDSAQSGNECNKAIDGNQDTFWHTFYGANGDPKPPHTYTIDMKSTQNVNGLSMLPRQDGSRNGWIGRHEVYLSTDGTNWGSPVAAGSWFADSTTKYSNFETRPARYVRLVAVTEASGQPWTSIAEINVFQASSYTAPQPGLGRWGPTIDLPIVPAAAAVEPTSGRVLVWSSYRNDAFGGSPGGVTLTSSWDPSSGIVSDRTVTVTKHDMFCPGISMDGNGQIVVTGGNDAKKTSLYDSSSDSWIPGPDMQVARGYQSSATMSDGRVFTIGGSWSGGIFEKNGEVYSPSSKTWTSLPNAKVNPMLTADKQGVYRSDNHAWLFGWKKGSVFQAGPSTAMNWYYTSGSGDVKSAGKRQSNRGVAPDAMCGNAVMYDAVRGKILTFGGSPDYQDSDATANAHIITLGEPGSTPNTVFASNGLYFARTFHTSVVLPDGSTFITGGQRRGIPFEDSTPVFTPEVYVPEQDTFYKQNPNSIVRVYHSISLLLPDGRVFNGGGGLCGDCTTNHFDAQIFTPNYLYDGNGNLATRPKITRTSTQSVKVGGRVTISTDSSIVKASLIRYGTATHTVNTDQRRIPLTLTNNGGNSYSFQVPSDSGIALPGYWMLFVMNSAGVPSVAATIRVTQ

SEQ ID NO: 12 - protein, 682 aa, Fusarium longipes
MKQLLTLALCFSSINAVAINPHGHHNNKGTGDHEGSLQFLSLRASAPLGSAIARENWVVTCDSAQPGNECNKAIDGDTNTFWHTFYGNNGDPKPPHTYTIDMGSNRNVNGLSVLPRQDGNRNGWIGRHEVYLSTDGSNWGSPVAYGSWFADSTTKYSNFETRPARYVRLVALTEASGQPWTSIAEINVYQAGSYTAPQAGLGRWGPTIDLPIVPAAAAIEPTSGRVLMWSSYRNDAFGGSPGGITLTSSWDPSSGIVSDRTVTVTNHDMFCPGISMDGNGQIVVTGGNDAKKTSLYDSSSDSWIPGPDMQVARGYQSSATMSDGRVFTIGGSWSGGIFEKNGEVYSPSSKTWTSLPGAKVNPMLTADKQGLYRSDNHAWLFGWKKGSVFQAGPSTAMNWYYTSGSGDVKSAGRRQSNRGLDPDAMCGNAVMYDAVAGKILTFGGSPDYQDSDATTNAHIITLGEPGSTPNTVFASNGLYFPRTFHTSVVLPDGSTFITGGQRRGIPFEDSTPQLTPEIYVPEQDTFYKQNPNSIVRVYHSISLLLPDGRVFNGGGGLCGDCTTNHFDAQIFTPNYLYNSNGDLATRPKITRTSAQSVRVGGRITISTDSSIRRASLIRYGTATHTVNTDQRRIPLTLTNNGGNSYSFQVPSDSGIALPGYWMLFVMNSAGVPSVATTLRVTR

SEQ ID NO: 13 - protein, 679 aa, Fusarium sambucinum [sequence omitted]

SEQ ID NO: 14 - protein, 639 aa, Fusarium sp.
ASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVASGSWFADSTTKYSNFETRPARYVRLVAITEANGQPWTSIAEINVFQASSYTAPQPGLGRWGPTIDLPIVPAAAAIEPTSGRVLMWSSYRNDAFGGSPGGITLTSSWDPSTGIVSDRTVTVTKHDMFCPGISMDGNGQIVVTGGNDAKKTSLYDSSSDSWIPGPDMQVARGYQSSATMSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMSGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSISKASLIRYGTATHTVNTDQRRIPLTLTNNGGNSYSFQVPSDSGVALPGYWMLFVMNSAGVPSVASTIRVTQ

SEQ ID NO: 15 - protein, 679 aa, Fusarium avenaceum
MKSLLTLALCFSSVLAVAITQPHKATQAKTHEGSLQFLSLRASAPIGSAINRNNWAVTCDSQQPGNECSKAIDGNRDTFWHTTWGSSDPKPPHTYTIDMKSTQNVNGISMLPRQDGSPNGWIGRHNVFLSTDGKNWGSAVATGTWYADNTEKYSNFETRPVRYVRLVAVTEANNQPWTSIAEINVFKADSYTKPQTGIGSWGPTLDFPIVPVAAAIEPTSGKVLVWSSYRNDAFGGSPGGVTLTSTWDPSTNVISQRTVTITKHDMFCPGISLDGTGQIVVTGGNDAQKTSLYDSSSDDWTPGPNMQVARGYQSSATLSNGRVFTIGGSWSGGSFEKNGEVYDPSSKTWTSLPGALVKPMLTADKQGIYRADNHGWLFGWKKGSVFQAGPSTAMNWYFTSGKGDVKPAGKRQSDRGIAPDAMCGNSVMYDAVKGKILTFGGSPDYQDADATTNAHIITIDEPGKTPKTVFASNGLYYARTFHTSVVLPDGSVFITGGQQHGVPFADTTPQLTPELYVPDADTFYKQQPNSIVRVYHSISLLLPDGRVFNGGGGLCGDCTTNHFDAQIFTPNYLYNNDGTLATRPKITKVSTKSVKVGGKVTVSTNGTIKQASLVRYSTATHTVNTDQRRVPLSLTNTGGNSYSFQLPNDSGIVLPGYWMLFVMNSAGVPSVASTLFVTQ

SEQ ID NO: 16 - protein, 681 aa, Fusarium subglutinans
MKSFWTLAFYLGNANAVAISQPASKAETPEGSLQFLSLRASAPIGTAINRDKWKVTCDSQHQGDECSKAIDGDRNTFWHTNWAAGATNDPKPPHTITIDMGSSQNVNGLSVLPRQDGSDHGWIGRHNVFLSTDGKNWGNAVATGTWFADITEKYSNFETRPARYVRLVAVTEANDQPWTSIAEINVFNAASYTSPQPGLGLWGPTLDFPIVPVAAAVEPTSGKVLVWSSYKNDAFGGSPGGVTLTSTWDPSTGVISQRTVTVTKHDMFCPGISMDGNGQVVVTGGNNAEKTSLYDSSSDSWIPGPDMKVARGYQFSATLSNGRVFTIGGSWSGGTFEKNGEVYDPSSKTWTSLPGALVKPMLTADQQGIYRSDNHGWLFGWKKGSVFQAGPSTAMNWYYTSGKGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCSTNHFDAQIYTPNNLYDSNGKLATRPKITNVSAKSAKVGGKITISTDTSIKQASLIRYGTSTHTVNTDQRRIPLSLRSTGSGNSYSFQVPSDSGIALPGYWMLFVMNSAGVPSIASTLLVTQ

SEQ ID NO: 17 - protein, 669 aa, Fusarium culmorum
MKHLLSLALCFTSINAVAVTIPHKSAGTGSPEGSLQFLSLRASAPIGSAISRNNWAVTCDSAQSGNECNKAIDGNKDTFWHTFYGANGDPKPPHTYTIDMKTTQNVNGLSMLPRQDGNQNGWIGRHEVYLSSDGTNWGSPVAAGSWFADSTTKYSNFETRPARYVRLVAVTEANGQPWTSIAEINPGLGRWGPTIDLPIVPAAAAIEPTSGRVLMWSSYRNDAFGGSPGGITLTSSWDPSSGIVSDRTVTVTKHDMFCPGISMDGNGQIVVTGGNDAKKTSLYDSSSDSWIPGPDMQVARGYQSSATTSDGRVFTIGGSWSGGVFEKNGEVYSPSSKTWTSLPNAKVNPMLTADKQGLYRSDNHAWLFGWKKGSVFQAGPSTAMNWYYTSGSGDVKSAGKRQSNRGVAPDAMCGNAVMYDAVKGKILTFGGSPDYQDSDATTNAHIITLGEPGTSPNTVFASNGLYFARTFHTSVVLPDGSTFITGGQRRGIPFEDSTPVFTPEIYVPEQDTFYKQNPNSIVRVYHSISLLLPDGRVFNGGGGLCGDCTTNHFDAQIFTPNYLYNSNGNLATRPKITRTSTQSVKVGGRITISTDSSSTKASLIRYGTATHTVNTDQRRIPLTLTNNGGNIYSFQVPSDSGVALPGYWMLFVMNSAGVPSVASTIRVTQ

SEQ ID NO: 18 - protein, 680 aa, Fusarium sp. FIESC_12
MRHLLTLALCFTSINAVAITKSHKSAGTGEPEGSVQFLSLRASAPIGTTISRDKWSVTCDSFHDGEGCDKAIDGDRNTFWHSQWGNGNDPKPPHTYTIDMGSTQNVNGLSVLPRQDGSSNGWIGRHEVFLSSDGSNWGSAVATGTWYADSTTKYSNFETRPARYVRIVALTEAKGQPWTSIAEINVFKADSYSAPQAGLGRWGPTLDFPIVPAAAYVEPTSGKVVVFSSYRNDAFGGSPGGITLTSTWDPASNVISERTVTVTKHDMFCPGISMDGTGQIVVTGGNDAKKTSLYDSSSDSWIPGPDMQVARGYQSSATTSDGRVFTIGGSWSGGIFEKNGEIYNPSSKTWTSLPGAKVNPMLTADRQGLYRSDNHAWIFGWKKGTVFQAGPSTAMNWYYTSGSGDVKSAGKRQSNRGVATDAMCGNAVMYDAVQGKILTFGGAKDYQDTDATTDAHIITLGEPGTTPNTVYASNGLWYPRTFHTSVVLPDGSTFITGGQVRGIPFEDSTPQLTPELYVPGDDTFYKQQPNSIVRVYHSVSLLLPDGRVFNGGGGLCGDCTTNHFDAQIFTPNYLYDNNGNLATRPKITNTSTKSVKVGGRVTITTDGSIQKASLVRYGTATHTVNTDQRRIPLTLSSSGRNSYSFTVPNDSGVALPGYWMLFVMNSAGVPSVSTTIRITQ

SEQ ID NO: 19 - protein, 681 aa, Fusarium oxysporum f. sp. cubense
MKSLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITINEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 20 - protein, 681 aa, Fusarium oxysporum Fo47
MKSFWTLALYLGSASAVAISQPAAKVETPEGSLQFLSLRASPPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 21 - protein, 681 aa, Fusarium sp. FOSC 3-a
MKSLWTLALYLGSVSAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKPATRPKITKVSAKSVKVGGKITITADTSIKQASLIRYGTSTHTVNTDQRRIPLSLRRTGTGNSYSFQVPSDSGIALPGYWMLFVMNSAGVPSVASTLLITQ

SEQ ID NO: 22 - protein, 666 aa, Fusarium oxysporum
MDLPAVRLIKVETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYASPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 23 - protein, 681 aa, Fusarium oxysporum f. sp. radicis-lycopersici 26381
MKSFWTLALYLGSASAVAISQPAAKVETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 24 - protein, 681 aa, Fusarium oxysporum f. sp. lycopersici 4287
MKPLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAPSYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGDVKSAGKRRSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 25 - protein, 681 aa, Fusarium oxysporum f. sp. cubense race 1
MKSLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 26 - protein, 681 aa, Fusarium oxysporum
MKSIWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 27 - protein, 681 aa, Fusarium oxysporum f. sp. raphani 54005
MKSLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGGVKSAGKRQSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 28 - protein, 681 aa, Fusarium oxysporum f. sp. pisi HDV247
MKSLWTLALYLGSASAVAISQPAAKAETPEGSLQFLSLRASAPIGTAINRDKWRLTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 29 - protein, 681 aa, Fusarium nygamai
MKSFWTLAFYLGSASAVAISQPAAKSETPAGSLQFLSLRASAPLGTAINRDKWQVTCDSQHEGDECSKAIDGDRNTFWHTNWAAEGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYKNDEFGGSPGGVTLTSTWDPSTGVISQRTVTNTKHDMFCPGISMDGNGQVVVTGGNNAEKTSLYDSSSDSWIPGPDMKVARGYQSSATLSNGRVFTIGGSWSGGRFEKNGEVYDPSSKTWTSLPGALVKPMLTADQQGIYRSDNHGWLFGWKKGSVFQAGPSTAMNWYYTGGKGDTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSVSLLLPDGRVFNGGGGLCGGCTTNHFDAQIYTPNNLYDSNGKLATRPKITKISAKSVKVGGKITISTDTSIKQASLIRYGTSTHTVNTDQRRIPLSLRSTGSGNSYSFQVPSDSGIALPGYWMLFVINSAGVPSVASTLLITQ

SEQ ID NO: 30 - protein, 681 aa, Fusarium oxysporum f. sp. cubense race 4
MKSLWTLALYLGSASAVAISQPAAKAETPEGSLQFLSLRASAPIGTAINRDKWRVTCDSQHEGDECSKAIDGDRDTFWHTAWAAGATNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPKALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLARRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 31 - protein, 681 aa, Fusarium oxysporum f. sp. vasinfectum 25433
MKSLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVTVTEANDQPWTSIAEINIFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSARSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 32 - protein, 681 aa, Fusarium mangiferae
MKSFWTLALYLGSASAVAITQPASKAETPEGSLQFLSLRASAPIGTAINRDKWKVTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYASPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGNGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTDISIKQASLIRYGTSTHTVNTDQRRIPLSLRRAGSGNSYSFQVPSDSGIALPGYWMLFVINSAGVPSVASTLVVTQ

SEQ ID NO: 33 - protein, 681 aa, Fusarium oxysporum
MKPLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVTVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGDVKSAGKRQSSRGTDPDAMCGNAVMYDAVKGKILTFGGSPSYQDSDATTNAHIITISEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 34 - protein, 681 aa, Fusarium verticillioides 7600
MKSFWTLAFYLGGASAVAISQPAAKSETPAGSLQFLSLRASAPLGTAINRDKWKVTCDSQHEGDECSKAIDGDRNTFWHTNWAAGGTNDPKPPHTITIDMGSSQNVNGLSVLPRQDGSNHGWIGRHNVFLSTNGKNWGDAVATGTWFADNTEKYSNFETRPARYVRLVAVTEANDQAWTSIAEINVFKAASYTSPQPGLGRWGPTLDFPIVPVAAAVEPTSGKVLVWSSYKNDEFGGSPGGVTLTSTWDPSTGVISQRTVTNTKHDMFCPGISMDGNGQVVVTGGNNAEKTSLYDSSSDSWIPGPDMKVARGYQSSATLSNGRVFTIGGSWSGGISEKNGEVYDPSSKTWTSLSGALVKPMLTADQQGLYRSDNHGWLFGWKKGSVFQAGPSTAMNWYYTSGNGNTKSAGKRQSSRGNDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSVSLLLPDGRVFNGGGGLCGGCTTNHFDAQIYTPNNLYDSNGKLATRPKITKVSAKSVKVGGKITISTDSSIKQASLIRYGTSTHTVNTDQRRIPLSLRSTGSGNSYSFQVPSDSGIALPGYWMLFVMNSAGVPSVASTLLVTQ

SEQ ID NO: 35 - protein, 681 aa, Fusarium oxysporum Fo5176
MKSLWTLALYLGSASAVAISQPAAKAETPEGSLQFLSLRASAPIGTAINRDKWRVTCDSQHEGDECSKAIDGDRDTFWHTAWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNDAQKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTTGNGGVKSAGKRQSSRGTDPDAMCGNAVMYDAVKGKILTFGGSPSYQDSDATTNAHIITISEPGSTPKTVFASNGLYYPRTFLTSVVLPDGNVFITGGQQRGIPFADSTPQLTPELYVPNDDTFYKQQPNSIVRVYHSISLLLPDGRVFNGGGGLCGDCDTNHFDAQIYTPNNLYDSNGKLATRPKITKVSAKSVKVGGKITITADTSIKQASLIRYGTSTHTVNTDQRRIPLSLRRTGTGNSYSFQVPSDSGIALPGYWMLFVMNSAGVPSVASTLLVTQ

SEQ ID NO: 36 - protein, 681 aa, Fusarium verticillioides
MKSFWTLAFYLGGASAVAISQPAAKSETPAGSLQFLSLRASAPLGTAINRDKWKVTCDSQHEGDECSKAIDGDRNTFWHTNWAAGGTNDPKPPHTITIDMGSSQNVNGLSVLPRQDGSNHGWIGRHNVFLSTNGKNWGDAVATGTWFADNTEKYSNFETRPARYVRLVAVTEANDQAWTSIAEINVFKAASYTSPQPGLGRWGPTLDFPIVPVAAAVEPTSGKVLVWSSYKNDEFGGSPGGVTLTSTWDPSTGVISQRTVTNTKHDMFCPGISMDGNGQVVVTGGNNAEKTSLYDSSSDSWIPGPDMKVARGYQSSATLSNGRVFTIGGSWSGGISEKNGEVYDPSSKTWTSLSGALVKPMLTADQQGLYRSDNHGWLFGWKKGSVFQAGPSTAMNWYYTSGNGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSVSLLLPDGRVFNGGGGLCGGCTTNHFDAQIYTPNNLYDSNGKLATRPKITKVSAKSVKVGGKITISTDSSIKQASLIRYGTSTHTVNTDQRRIPLSLRSTGSGNSYSFQVPSDSGIALPGYWMLFVMNSAGVPSVASTLLVTQ

SEQ ID NO: 37 - protein, 681 aa, Fusarium proliferatum
MKSFWTFALYLGSASAVAISQPASKAETPEGSLQFLSLRASAPIGTAISRDKWKVTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVIVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVEGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 38 - protein, 681 aa, Fusarium verticillioides
MKSFWTLAFYLGGASAVAISQPAAKSETPAGSLQFLSLRASAPLGTAINRDKWKVTCDSQHEGDECSKAIDGDRNTFWHTNWAAGGTNDPKPPHTITIDMGSSQNVNGLSVLPRQDGSNHGWIGRHNVFLSTNGKNWGDAVATGTWFADNTEKYSNFETRPARYVRLVAVTEANDQAWTSIAEINVFKAASYTSPQPGLGRWGPTLDFPIVPVAAAVEPTSGKVLVWSSYKNDEFGGSPGGVTLTSTWDPSTGVISQRTVTNTKHDMFCPGISMDGNGQVVVTGGNNAEKTSLYDSSSDSWIPGPDMKVARGYQSSATLSNGRVFTIGGSWSGGISEKNGEVYDPSSKTWTSLSGALVKPMLTADQQGLYRSDNHGWLFGWKKGSVFQAGPSTAMNWYYTSGNGNTKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSVSLLLPDGRVFNGGGGLCGGCTTNHFDAQIYTPNNLYDSNGKLATRPKIIKVSAKSVKVGGKITISTDSSIKQASLIRYGTSTHTVNTDQRRIPLSLRSTGSGNSYSFQVPSDSGIALPGYWMLFVMNSAGVPSVASTLLVTQ

SEQ ID NO: 39 - protein, 681 aa, Fusarium proliferatum; identical to SEQ ID NO: 37

SEQ ID NO: 40 - protein, 681 aa, Fusarium proliferatum ET1
MKSFWTFALYLGSASAVAISQPASKAETPEGSLQFLSLRASAPIGTAISRDKWKVTCDSQHEGDECSKAIDGDSNTFWHTDWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNNAEKTSLYDSSRDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 41 - protein, 681 aa, Fusarium proliferatum
MKSLWTFAFYLGSASAVAISQPASKAETPEGSLQFLSLRASAPIGTAINRDKWKVTCDSQHEGDECSKAIDGDRNTFWHTNWAAGGTNDPKPPHTITIDMGSSQNVNGLSVLPRQDGSDHGWIGRHNVLLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGNGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDPGIALPGYWMLFVINSVGVPSVASTLLVTQ

SEQ ID NO: 42 - protein, 681 aa, Fusarium fujikuroi
MKSFWTLALYLGSASAVAISQPASKAETPEGSLQFLSLRASAPIGTAISRDKWKVTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGSGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 43 - protein, 681 aa, Fusarium fujikuroi IMI 58289
MKSFWTLALYLGSASAVAISQPASKAETPEGSLQFLSLRASAPIGTAISRDKWKVTCDSQHEGDECSKAIDGDRNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGSGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKEGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 44 - protein, 681 aa, Fusarium fujikuroi
MKSFWTLALYLGSASAVAISQPASKAETPEGSLQFLSLRASAPIGTAISRDKWKLTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYIRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGSGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 45 - protein, 681 aa, Fusarium proliferatum
MKSFWTFALYLGSASAVAISQPASKAETPEGSLQFLSLRASAPIGTAISRDKWKVTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTATVTKHDMFCPGISMDGNGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGVALPGYWMLFVINSAGVPSVASTLLVTQ

SEQ ID NO: 46 - protein, 681 aa, Fusarium fujikuroi
  misc_feature (55)..(55): Xaa can be any naturally occurring amino acid
MKSFWTLALYLGSASAVAISQPASKAETPEGSLQFLSLRASAPIGTAISRDKWKXTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYIRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGSGQVVVTGGNNAEKTSLYDSSSDSWIPGPDMKVARGYQSSATLSNGRVFTIGGSWSGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGNSYSFQVPSDSGIALPGYWMLFVINSAGVPSVASTLLVTQ

SEQ ID NO: 47 - protein, 684 aa, Fusarium euwallaceae
MRSPILTLCLAGLFWDEAMAIVIPAQNSTKVKVQMEHLSLRATGPLGTAINRNNWKVTCDSDNPDSDNACQKAIDGDVNSFWHTAWFEDTSKDPGLPHTITVDMKTVQNVNGISALPRQDGTTHGWIARHDIFLSTDGKTWGSPVATGTWYADGTEKFSNFETKRARYVRIVAITEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRTFKPGEIFDPSSRKWTLLQNAKVEPMLTADAQGIFRSDNHAWLFGWKGETIFQAGPSSAMNWYYTQGKGTVKPAGKRQSSRGVDPDSMCGNAVMFDAVKGKIVTFGGTPNYQDSYATTNAHIITIGAPGTQASVAFASNGMYYPRVFHTSVLLPDGTVFITGGQEYAIPFEDSTPQLTPEMYIPDSDTFIKQQPNSIVRTYHSMSILLPDARVFNGGGGLCGDCSTNHFDAQIFTPNYLLTKDGKPATRPKIVSVSATTIKVGGSITVTTGGVVNTASLIRYGTATHTVNTDQRRIPLTLTGAGKNKYTFKVPGDSGIALPGYWMLFVMNSAGVPSVASTIKVTT

SEQ ID NO: 48 - protein, 684 aa, Fusarium ambrosium
MRSPILTLCLAIFFWDEAMAIVIPAQNSTKVKVQMEHLSLRATGPLGTAINRNNWKVTCDSDNPDSDNACQKAIDGDVNSFWHTAWFEDTSKDPGLPHTLTVDMKTVQNVNGISALPRQDGTTHGWIARHDIFLSRDGKAWGSPVATGTWYADGTEKFSNFETKRARYVRIVATTEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRTFKPGEIFDPSSRKWTLLSNAKVEPMLTADAQGIFRSDNHAWLFGWKGETIFQAGPSSAMNWYYTQGKGTVKPAGKRQSSRGVDPDSMCGNAVMFDAVKGKIVTFGGTPNYQDSYATTNAHIITIGAPGTQASVAFASNGMYYPRVFHTSVLLPDGTVFITGGQEYAIPFEDSTPQLTPEMYIPDSDTFIKQQPNSIVRTYHSMSILLPDARVFNGGGGLCGDCSTNHFDAQIFTPSYLLNKDGKPATRPKIVSVSATTIKVGGSITVTTGGVVNTASLIRYGTATHTVNTDQRRIPLTLTGAGKNKYTFKVPGDSGIALPGYWMLFVMNSAGVPSVASTIKVTT

SEQ ID NO: 49 - protein, 684 aa, Fusarium sp. AF-3
MWSPILTLCLAGIFWDEAMAIVIPAQNSTKVKVQMEHLSLRATGPLGTAINRNNWKVTCDSDNPDSDNACQKAIDGDVNSFWHTAWFEDTSKDPGLPHTLTVDMKTVQNVNGISALPRQDGTTHGWIARHDIFLSTDGKTWGSPVATGTWYADGTEKFSNFETKRARYVRIVATTEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRAFKPGEIFDPSSRKWTLLSNAKVEPMLTADAQGIFRSDNHAWLFGWKGETIFQAGPSSAMNWYYTQGKGTVKPAGKRQSSRGVDPDSMCGNAVMFDAVKGKIVTFGGTPNYQDSYATTNAHIITIGAPGTQASVAFASNGMYYPRVFHTSVLLPDGTVFITGGQEYAIPFEDSTPQLTPEMYIPDSDTFIKQQPNSIVRTYHSMSILLPDARVFNGGGGLCGDCSTNHFDAQIFTPSYLLTKDGKPATRPKIVSVSATTIKVGGSITVTTGGVVNTASLIRYGTATHTVNTDQRRIPLTLTGAGKNKYTFKVPGDSGIALPGYWMLFVMNSAGVPSVASTIKVTT

SEQ ID NO: 50 - protein, 684 aa, Fusarium kuroshium
MWSPILTLCLAGLFWDEAMAIVIPAQNSTKVKVQMEHLSLRATGPLGTAINRNNWKVTCDSDNPDSDNACQKAIDGDVNSFWHTAWFEDTSKDPGLPHTLTVDMKTVQNVNGISALPRQDGITHGWIARHDIFLSTDGKTWGSPVATGTWYADGTEKFSNFETKRARYVRIVATTEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRTFKPGEIFDPSSRKWTLLQNAKVEPMLTADAQGIFRSDNHAWLFGWKGETIFQAGPSSAMNWYYTQGKGTVKPAGKRQSSRGVDPDSMCGNAVMFDAVKGKIVTFGGTPNYQDSYATTNAHIITIGAPGTQASVAFASNGMYYPRVFHTSVLLPDGTVFITGGQEYAIPFEDSTPQLTPEMYIPDSDTFIKQQPNSIVRTYHSMSILLPDARVFNGGGGLCGDCSTNHFDAQIFTPSYLLTKDGKPATRPKIVSVSATTIKVGGSITVTTGGVVNTASLIRYGTATHTVNTDQRRIPLTLTGAGKNKYTFKVPGDSGIALPGYWMLFVMNSAGVPSVASTIKVTT

SEQ ID NO: 51 - protein, 684 aa, Fusarium sp. AF-6
MRSSILTFCLAGLFWDEAMAIVIPTQNSTKVKVQMEHLSLRATGPLGTAINRSNWKVTCDSDNPDSDNACQKAIDGDVNSFWHTAWFEDTSKDPGLPHTLTVDMKTVQNVNGISALPRQDGITHGWIARHDIFLSTDGKTWGTPVATGTWYADGTEKFSNFETKRARYVRIAAVTEAYDGPWTSIAEFNVYKAATYTAPKSGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRAFKPGEIFDPSSRKWTLLQNAKVEPMLTADAQGIFRSDNHAWLFGWKGETIFQAGPSSAMNWYYTQGKGTVKPAGKRQSSRGVDPDSMCGNAVMFDAVKGKIVTFGGTPNYQDSYATTNAHIITIGAPGTQASVAFASNGMYYPRVFHTSVLLPDGTVFITGGQEYAIPFEDSTPQLTPEMYIPDSDTFIKQQPNSIVRTYHSMSILLPDARVFNGGGGLCGDCSTNHFDAQIFTPSYLLTKDGKPATRPKIVSVSATTIKVGGSITVTTGGVVNTASLIRYGTATHTVNTDQRRIPLTLTGAGKNKYTFKVPGDSGIALPGYWMLFVMNSAGVPSVASTIKVTT

SEQ ID NO: 52 - protein, 684 aa, Fusarium sp. AF-8
MWSPILTLCLTGLFWDEAMAIVVPAQNSTKVKVQMEHLSMRATGPLGTAINRNNWKVTCDSDNPDSDNACQKAIDGDVNSFWHTAWFEDTSKDPGLPHTLTVDMKTVKNVNGISALPRQDGTTHGWIARHDIFLSTDGKTWGSPVATGTWYADGTEKFSNFETKRARYVRIVAITEAYDGPWTSIAEFNVYKAATYTAPKTGIGMWGPTLDFPIVPVAGAVDPGTGKVLVWSSYYHDTMNGSPGGMTLTSLWDPETGIITQREVFETNHDMFCPGISMDGTGQIVVTGGNNAARTSTWDPVKNQWVSAPDMKIPRGYQSSATTSNGKVFTIGGSWGGDRAFKPGEIFDPKSRKWTLLPNAKVEPMLTADAQGIFRSDNHAWLFGWKGETVFQAGPSSAMNWYYTQGKGTVKPAGKRQSSRGVDPDSMCGNAVMFDAAKGKIVTFGGTPNYQDSYATTNAHIITIGAPGTQASVAFVSDGMYYPRVFHTSVLLPDGTVFITGGQEYAIPFEDSTPQLTPELYIPDSDTFIKQQPNSIVRTYHSMSILLQDARVFNGGGGLCGDCSTNHFDAQIFTPSYLLTKDGKPAARPKIVSVSATTIKVGGSITVTTGGVVNTASLIRYGTATHTVNTDQRRIPLTLASAGKNKYTFKVPGDSGIALPGYWMLFVMNSAGVPSVATTIKVTT

SEQ ID NO: 53 - protein, 681 aa, Fusarium fujikuroi
  misc_feature (330)..(330): Xaa can be any naturally occurring amino acid
  misc_feature (332)..(332): Xaa can be any naturally occurring amino acid
MKSFWTLALYLGSASAVAISQPASKAETPEGSLQFLSLRASAPIGTAISRDKWKVTCDSQHEGDECSKAIDGDSNTFWHTNWAAGGTNDPKPPHTITIDMGSSQNVNGLSVLPRQDGSDHGWIGRHNVFLSTDGKNWGDAVATGTWFADNTEKYSNFETRPARYVRLVAVTEANDQPWTSIAEINVFKAASYTSPQPGLGRWGPTLDFPIVPVAAAVEPTSGKVLVWSSYRNDAFGGSPGGVTLTSTWDPSTGVISQRTVTVTKHDMFCPGISMDGDGQVVVTGGNNAEKTSLYDSSSDSWIPGPDMKVARGYQSSATLSNGRVFTIGGXWXGGIFEKNGEVYDPSSKTWTSLPGALVKPMLTADQQGLYRSDNHGWLFGWKKGSVFQAGPSTAMNWYYTSGSGATKSAGKRQSSRGTDPDAMCGNAVMYDAVKGKILTFGGSPSYQDSDATTNAHIITIGEPGSTPKTVFASNGLYYPRTFHTSVILPDGNVFITGGQQRGIPFADSTPQLTPELYVPNDDTFYKQQPNSIVRVYHSISLLLPDGRVFNGGGGLCGDCDTNHFDAQIYTPNNLYDSSGKLATRPKITKVSAKSVKVGGKITISTNTSIKQASLIRYGTSTHTVNTDQRRIPLSLRSTGSGSSYSFQVPSDSGIALPGYWMLFVINSAGVPSVASTLLVTQ

SEQ ID NO: 54 - protein, 674 aa, Pochonia chlamydosporia 170
MKFTAETVLLATLFAGHVAALVPRKIKLKQHYRENSTFAKLWAAAPIGNEINRAGWKVTCDSFEPGNECGKAIDGNNNSFWHTKYDGSNLPHQIVVDFGTVKNVNGISALPRQDDNDHGFIAQHDVSVSTDGKTWESVAGGNWYGGDKTLKFANFETRSIRYVRLKATSEVNGNPWTSLAEIKAYEAKAGPTAYNQNGKWGPTINFPTVPVAATVDPNSGKVIVWSSYTYDNYLGSSQDRVFTSIWDPKTGIVTPKLVDNTDHDMFCPGISIDGTGKMVVTGGNSAQKTTLYDFASQTWIPGPDMNLPRGYQASATLSDGRVFTIGGCWSGGWFEKNGEVYDPKAKTWKNLSGALVKPMLTNDAQGIYRADNHGWFFGWKSGSVFQAGPSTAMNWYSTTGNGGVTPAGQRKSDRGVDGDSMNGNAVMYDATQGKILTFGGAPSYQDSDATAHAHLITIGNPGTQAKVQFASNGLWSARSFHTSVVLPDGNVFITGGQSYAVPFSDDTPQLTPELYKPADDSFYQQQPNSIIRVYHSISLLLPDGRVLNAGGGLCGDCNTNHFDGQIFTPQYLLTSTGQPATRPVIKSATLSGRKITISTDSAVSTASLIRFGTATHTVNTDQRRIPLTLTKTGTNMYTANAPTDSGILLPGYYMLFVMNANGVPSVSKTLSFLV

SEQ ID NO: 55 - protein, 1067 aa, Pochonia chlamydosporia 170
MKPVYALTICLGLLEGADAIAIPEFVSPKVGYSTSLNHGEKKAESGSDANSSPRVFGAYGPKSKLVGLPPVRRSDKTEESYGSDDGVDDDDQDDSGDNTADPPEDSPDSDDPELIYPEPMMAPAYVHTGVLPVDKPEEAPKSKSSKVDTKSKSKSGSKPDDTVHTAKHGGYDDSESGSSGKKKQKGKDKNKGNDDNPGKHSGDDDASKSRTKSEDSKPTSKSTTSNTSTSGTAPLPTSVNTRPTTGRHSGDDDDKKSRSKTEDSPRSGSPKPTSTSDASSKGSGKGKGKGNDGSGKHSGDDDATKTSSKKGSKDDSAKADDSKSAKPAFKKSSSTPRHPRGITATHQSGSTFDPNSAKGGGKNGPGFFKPFEPPKLNNGYIQAVPPQASQKNSKSSPKVKPKPRLNGKVSGPFSSLRKREQLLSLRAAAPLESVTIDRSKWKATCDSVHEGDECQNAIDGNGDTIWHTQWEGTEPAPPHMITVDMKKVYNVNGISMLPRQDGSQNGYIAQHQVFLSKDNHNWGSPVAYGTWYSDYTTKYANFDTQPARYVRLVAITEANGGPWTSIAELNVYQGNDYTAPQPSSLGAWGPTVNFPVIPVAGTVDPKTGKVLIWSSWARDTMDGGPGGLTLTSMWDPATGLVAERDVTETNHDMFCPGISLDGNGQLVVTGGNNAERTSLFDPVKQAWIPGPDMKVARGYHASATTSTGKVFTIGGSWSGGEYFKNGEVYDPKKKTWTLLNKADVQKMLTADTQGLFRSDNHAWLFGWKKGTVFQAGPSQAMNWYYTDGKDGNVKTAGKRQSDRGVAPDAMCGNAIMFDAVKGKILTNGGTPNYQDSDATADAHIITINNPGNKADVSYASEGLYHARVFHSSVVLPNGNVFITGGQEYAIPFADSMPVLEPEMYLPDEDRFVLMKPNNIVRTYHSIALLLPDGRVFNGGGGLCGGCDTNHFDAQLYTPPYLYDSKGRLATRPKIASVSVSTVKVGGTVTVQTSGAVVRASLVRYGTATHTVNSDQRRVPLTLSNAGKNSYSFQVPSDPGVALPGYWMLFVMDKNGVPSVASTIKVTG

SEQ ID NO: 56 - protein, 1067 aa, Fusarium graminearum; identical to SEQ ID NO: 55

SEQ ID NO: 57 - protein, 1071 aa, Fusarium oxysporum
MKVVYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPPDDTPSDSSDDIDEVVYAEPFMQPTYVRFGPAPVDKPEVAPKAKSNSKDTASVAKHGGDDDSSSGGHKQKGKDKNKGKDDATKSRKKPEDSPKTSSNSATDSVSVTATSTAAPTSTAAPTSPPKGDDKGKSKGKGKGKGKDGGGRHNGDDDAAKSRTKVEDSTKTYSDDSGKGKGKGKGDGKGRHDGGVDATKSRAKAEDSETNSKKSSKKGSKDDSKSKPSKKSFITHQSGKTFDADSTKGGGKNGPGYNPSFDVPKLNKGYIQAIPPKASTINNKFSSRVKPRPQLNTEKVRPLSSSSSSSSSSLLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQIFLSKDGKTWGSPVAYGNWYSDWTVKYANFDTQPARFVKLVALTEANGNPWTSIAELNVFQANDYVPPQASQGAWGPTINFPIIPVAGTVDPNTGKVLVWSSWARDTMSGGPGGLTLTSTWDPATGQVAERQVTETNHDMFCPGISLDGNGQLVVTGGNNAERTSLFDPVKQAWVSGPNMKVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGVAPDAMCGNAIMFDAVKGKILTHGGTPNYQDSDATTDAHIITVGNPGANVSVAYASEGLFFPRVFHSSVVLPNGNVFITGGQQYAVPFEDSTPQLQPEMYYPDRDGFELMKPNNIVRTYHSIALLLPDGRVFNGGGGLCGGCDTNHFDAQLYTPPYLYDSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 58 - protein, 947 aa, Fusarium graminearum PH-1
MAPAYVHTGVLPVDKPEEAPKSKSSKVDTKSKSKSGSKPDDTVHTAKHGGDDDSESGSSGKKKQKGKDKNKGNGDNPGKHSGDDDASKSRTKSEDSKPTSKLTTSNTSTSGTAPLPTSVNTRPTTGRHSGDDDDKKSRSKTEDSPRSGSPKPTSTSDASSKGSGKGKGKGNDGSGKHSGDDDATKTSSKKGSKDDSAKADDSKSAKSAFKKSSSTPRHPRGITATHQSGSTFDPNSAKGGGKNGPGFFKPFEPPKLNNGYIQAVPPQASQKNSKSSPKVKPKPRLNGKVSGPFSSLRKREQLLSLRAAAPLESVTIDRSKWKATCDSVHEGDECQNAIDGNGDTIWHTQWEGTEPAPPHMITVDMKKVYNVNGISMLPRQDGSQNGYIAQHQVFLSKDNHNWGSPVAYGTWYSDYTTKYANFDTQPARYVRLVAITEANGGPWTSIAELNVYQGNDYTAPQPSSLGAWGPTVNFPVIPVAGTVDPKTGKVLIWSSWARDTMDGGPGGLTLTSMWDPATGLVAERDVTETNHDMFCPGISLDGNGQLVVTGGNNAERTSLFDPVKQAWIPGPDMKVARGYHASATTSTGKVFTIGGSWSGGEYFKNGEVYDPKKKTWTLLNKADVQKMLTADTQGLFRSDNHAWLFGWKKGTVFQAGPSQAMNWYYTDGKDGNVKTAGKRQSDRGVAPDAMCGNAIMFDAVKGKILTNGGTPNYQDSDATADAHIITINNPGNKADVSYASEGLYHARVFHSSVVLPNGNVFITGGQEYAIPFADSMPVLEPEMYLPDEDRFVLMKPNNIVRTYHSIALLLPDGRVFNGGGGLCGGCDTNHFDAQLYTPPYLYDSKGRLATRPKIASVSVSTVKVGGTVTVQTSGAVVRASLVRYGTATHTVNSDQRRVPLTLSNAGKNSYSFQVPSDPGVALPGYWMLFVMDKNGVPSVASTIKVTG

SEQ ID NO: 59 - protein, 684 aa, Pochonia chlamydosporia 123
MELFGAIAFVLCQLSCHVHAVTLKQTSTTALVHATEQAALHLLAAAPVGYRIDRANWKVTCDSQEIGYECGKAIDGDNSTVWHTAFRADNPKPPHNITVDMGSIQNVNGLSILPRQDSDSNGWVARHQVLVSADGQRWEHPVATGTWYADSTEKFSNFEPEQARFVRLVADSEVSGNPWTSVAELNVYKAGTDPAPAKSASSGKWGPTLDFPIVPVAAAVVPQSGKVLVWSAYENDKFEGSPGGYTLTSTWDPATGDVTQRNVTNIGHDMFCPGISMDSNGQIVVTGGNNAQKTSFYDAASDSWVPGPDMTVSRGYQSSATCSDGRIFTIGGSWSGGQFEKNGEIYDPSTKTWKMLSLAAVKPMLTADKKGRYRADNHGWLFGWRNGTVFQAGPSTAMNWYFTGGDGGVKSAGSRKSSRGPDPDSMCGNAVMFDATNGKILTVGGSPWYQDNDATANAHIITVRGPGSEPSVSFAGNGMHHARIFASSVVLPDGSVFITGGQQHSIPFADSTPQFTPELYDPEKDQFLEQAPNSIVRVYHSLALLLPDATVFNGGGGLCGGSCATNHFDAQIYSPRYLFDHDGSPAVRPVIKSVSRTSVKPGDSLSFTTDSAVQHASLVRYGTATHTVNTDQRRVPLTLDASGANTYAVQLPNDPGIMLPGYWMLFVMNERGTPSVAASIKAHL

SEQ ID NO: 60 - protein, 1073 aa, Fusarium oxysporum f. sp. cepae
MKVVYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPPDDTPSGSSDDIDEVVYAEPFMQPTYVRFGPAPVDKPEVAPKAKSNSKDTASVAKHGVDDDSSSGGHKQKGKDKNKGKDDATKSRKKPEDSPKTSSNSATDSVSVTATSTAAPTSPPKGDDKGKSKGKGKGKGKDGGGRHNGDDDATKSRTKVEDSTKTRSSAPTKTYSDDSGKGKGKGKGDGKGRHDGGVDATKSRAKAEDSETNSKKSSKKGSKDDSKSKPSKKSFITHQSGKTFDADSTKGGGKNGPGYNPSFDVPKLNKGYIQAIPPKASTINNKFSSRVKPRPQLNTEKVRPLSSSSSSSSSSLLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQIFLSKDGKTWGSPVAYGNWYSDWTVKYANFDTQPARFVKLVALTEANGNPWTSIAELNVFQANDYVPPQASQGAWGPTINFPIIPVAGAVDPNTGKVLVWSSWARDTMSGGPGGLTLTSTWDPATGQVAERQVTETNHDMFCPGISLDGNGQLVVTGGNNAERTSLFDPVQQAWVSGPNMQVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGIAPDAMCGNAIMFDAVKGKILTHGGTPNYQDSDATTDAHIITVGNPGANVSVAYASEGLFFPRVFHSSVVLPNGNVFITGGQQYAVPFEDSTPQLQPEMYYPDRDVFELMKPNNIVRTYHSIALLLPDGRVFNGGGGLCGGCDTNHFDAQLYTPPYLYDSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 61 - protein, 1074 aa, Fusarium oxysporum
MKVVYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPSDVTPPSPPDDTPSGSSDDIDKVVYAEPFMQPTYVRFGPAPVDKPEVAPKAKSNSKDTGSVAKHGGDDDSSSGGHKQKGKDDKGRHNGDDDATKSRKKPEDSPKTSSSSATDSVSVTATSTAAPTSPPTGDDKGKGKGKDGGGRHNGDDDATKSRTKVEDSTKTRSSAPTKTSSDDSGKGKGKGKGGGKGRHDGGDEATKSRAKAEDSKTNSKKSSKKGSKDDSKSKPSKKSFITHQSGKTFDADSTKGGGKNGPGYNPSFDVPKLNKGYIQAIPPKASTINNKFSSRVKPRPQLNTEKVRPLSSSSSSLLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQIFLSKDGKTWGSPVAYGNWYSDWTVKYANFDTQPARFVKLVALTEANGNPWTSIAELNVFQANDYVPPQASQGAWGPTINFPIIPVAGTVDPNTGKVLVWSSWARDTMSGGPGGLTLTSTWDPATGQVAERQVTETNHDMFCPGISLDGNGQLVVTGGNNAERTSLFDPVQQAWVSGPNMQVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGVAPDAMCGNAIMFDAVKGKILTHGGTPNYQDSDATTDAHIITVGNPGANVSVAYASEGLFFPRVFHSSVVLPNGNVFITGGQQYAVPFEDSTPQLQPEMYYPDRDGFELMKPNNIVRTYHSIALLLPDGRVFNGGGGLCGGCDTNHFDAQLYTPPYLYYSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 62 - protein, 1070 aa, Fusarium oxysporum
MKVVYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPPDDTPSGSSDDIDEVVYAEPFMQPTYVRFGPAPVDKPEVAPKAKSNSKDTASVAKHGVDDDSSSGGHKQKGKDKNKGKDDATKSRKKPEDSPKTSSNSATDSVSVTATSTAAPTSPPKGDDKGKSKGKGKGKGKDGGGRHNGDDDATKSRTKVEDSTKTRSSAPTKTYSDDSGKGKGKGKGDGKGRHDGGVDATKSRAKAEDSETNSKKSSKKGSKDDSKSKPSKKSFITHQSGKTFDADSTKGGGKNGPGYNPSFDVPKLNKGYIQAIPPKASTINNKFSSRVKPRPQLNTEKVRPLSSSSSSSLLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQIFLSKDGKTWGSPVAYGNWYSDWTVKYANFDTQPARFFKLVALTEANGNPWTSIAELNVFQANDYVPPQASQGAWGPTINFPIIPVAGTVDPNTGKVLVWSSWARDTMSGGPGGLTLTSTWDPATGQVAERQVTETNHDMFCPGISLDGNGQLVVTGGNNAERTSLFDPVKQAWVSGPNMKVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGIAPDAMCGNAIMFDAVKGKILTHGGTPNYQDSDATTDAHIITVGNPGANVSVAYASEGLFFPRVFHSSVVLPNGNVFITGGQQYAVPFEDSTPQLQPEMYYPDRDVFELMKPNNIVRTYHSIALLLPDGRVFNGGGGLCGGCDTNHFDAQLYTPPYLYDSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 63 - protein, 1071 aa, Fusarium oxysporum f. sp. narcissi
MKVVYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPPDDTPSGSSDDIDEVVYAEPFMQPTYVRFGPAPVDKPEVAPKAKSNSKDTASVAKHGVDDDSSSGGHKQKGKDKNKGKDDATKSRKKPEDSPKTSSNSATDSVSVTATSTAAPTSPPKGDDKGKSKGKGKGKGKDGGGRHNGDDDATKSRTKVEDSTKTRSSAPTKTYSDDSGKGKGKGKGDGKGRHDGGVDATKSRAKAEDSETNSKKSSKKGSKDDSKSKPSKKSFITHQSGKTFDADSTKGGGKNGPGYNPSFDVPKLNKGYIQAIPPKASTINNKFSSRVKPRPQLNTEKVRPVSSSSSSSSLLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQIFLSKDGKTWGSPVAYGNWYSDWTVKYANFDTQPARFVKLVALTEANGNPWTSIAELNVFQANDYVPPQATQGAWGPTINFPIIPVAGTVDPNTGKVLVWSSWARDTMSGGPGGLTLISTWDPATGQVAERQVTETNHDMFCPGISLDGNGQLVVTGGNNAERTSLFDPVKQAWVSGPNMKVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGVAPDAMCGNAIMFDAVKGKILTHGGTPNYQDSDATTDAHIITVGNPGANVSVAYASEGLFFPRVFHSSVVLPNGNVFITGGQQYAVPFEDSTPQLQPEMYYPDRDVFELMKPNNIVRTYHSIALLLPDGRVFNGGGGLCGGCDTNHFDAQLYTPPYLYDSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 64 - protein, 1083 aa, Fusarium oxysporum f. sp. cubense
MKAGYALTLCLGIADAVVLPVFSAPKGGYALPRPDSDDTTDSSKTAAGSKTNLDSMLPVFGKAKPKTGFPASALSRRVARIEQTYGSDDESTDDSDDTPTDPSDATPSSPSDVTPPSPPDDTPSGSSDDIDEVVYAEPFMQPTYVRFGPAPVDKPEVASKAKSNSKDTASVAKHGGDDDSSSGGHKQKGKDDKGRHNGDDDATKSRKKPEDSPKTSSSSATDSVSVTATSTAAPTSPPTGDDKGKGKGKGKGKGKDGGGRHNGDDDATKSQTKVDYSTKTRSSAPTKTSSDDSGKGKGKGKGGGKGRHDGGDDAAKSRAKAEDSKTNSKKSSKKGSKDDSKSKPSKKSFITHQSGKTFDADSTKGGGKNGPGYNPSFDAPKLNKGYIQAIPPKASTINNKFSSRVKPRPQLNTEKVRPLSSSSSSSSSLLKRDESKNILSLRAAAPFNSAAIDRNKWSVTCDSVHEGDDCKNAIDGNGDTMWHTQWEGSEPAPPHSITVDMKKSYNVNGISMLPRQDGSQNGYMAQHQVFLSKDGKTWGSPVAYGNWYNDWTVKYANFDTQPARFVKLVALTEANGNPWTSIAELNVFQANDYVPPQASQGAWGPTINFPIIPVAGTVDPNTGKVLVWSSWARDTMSGGPGGLTLTSTWDPATGQVAERQVTETNHDMFCPGISLDGNGQLVVTGGNNAERTSLFDPVKQAWVSGPNMQVARGYQSSATTSTGKVFTIGGSWSGGESFKNGEVYDPKKKTWTLLNKADVQKMLTNDAQGLFRSDNHAWLFGWKSGTVFQAGPSKNMNWYYTEKKNGDVKTAGQRASDRGVAPDAMCGNAIMFDAVKGRILTHGGTPNYQDSDATTDAHIITVGNPGANVSVAYASEGLFFPRVFHSSVVLPNGNVFITGGQQYAVPFEDSTPQLQPEMYYPDKDGFELMKPNNIVRTYHSIALLLPDGRVFNGGGGLCGGCDTNHFDAQLYTPSYLYDSKGKLATRPKITSVSVSTIKVGGTVTVQTGGAIVQASLVRYGTATHTVNSDQRRIPLTLANAGKNSYSFQVPSDPGVALPGYWMLFVIDKNGVPSVASTIKVTSS

SEQ ID NO: 65 - protein, 951 aa, Fusarium oxysporum f. sp. cubense race 4
MQPTYVRFGPAPVDKPEVAPKAKSNSKDTASVAKHGGDDDSNSGGHKQKGKDKNKGKDDKGRHNGDDDATKSRKKPEDSPKTSSSSATDTVSVTATSTAAPTSPPTGDDKGKGKGKGKGKGKDGGGRHNGDDDATKSRTKLEDSTKTRSSAPTKTSSDDSGKGKGKGKGGGKGRHDGGDDAAKSQAKAEDSKTNSKKSSKKGSKDDSKSKPSKKSFITHQSGKTFDADSTKGGGKNGPGYNPSFDVPKLNKGYIQAIPPKASTINNKFSSRVKPRPQLNTEKVRPLSSSSSSSFLKRDESKNILSLRAAAPFNSAAIDRKKWSVTCDSVHEGDDCKNAIDGNGDTMWHTQWEGSEPAPPHSITVDMKKSYNVNGISMLPRQDGSQNGYIAQHQVFLSKDGKTWGSPVAYGNWYNDWTVKYANFDTQPARFVRLVALTEANGNPWTSIAELNVFQANDYVPPQASQGAWGPTINFPIIPVAGTVDPNTGKVLVWSSWARDTMSGGPGGLTLTSTWDPATGQVAERQVTETNHDMFCPGISIDGNGQLVVTGGNNAERTSLFDPVKQAWVSGPNMQVARGYQSSATTSTGKVFTIGGSWSGGESFKNGEVYDPKKKTWTLLNKADVQKMLTNDAQGLFRSDNHAWLFGWKSGTSGTVFQAGPSKNMNWYYTEKKNGDAKTAGQRASDRGVAPDAMCGNAIMFDAVKGKILTHGGTPNYQDSDATTDAHIITVGNPGTNASVAYASEGLFFPRVFHSSVVLPNGNVFITGGQKYAVPFEDSTPQLQPEMYYPDKDGFELMKPNNIVRTYHSIALLLPDGRVFNGGGGLCGGCDTNHFDAQLYTPPYLYDSKGKLATRPKITSVSVSTIKVGGTVTVQTGGAIVQASLVRYGTATHTVNSDQRRIPLTLANAGKNSYSFQVPSDPGVALPGYWMLFVMDKNGVPSVASTIKVTGS

SEQ ID NO: 66 - protein, 676 aa, Metarhizium guizhouense ARSEF 977
MKLTTETVLLGALLAGQAAAGLVPRSFTVKQHYHENSTFSKLFAAPPIANGEIDRAGWKVTCDSFEPGNECSKAIDGNNDTFWHTKFEGSNVPHQIVVDFGSTHNINGISALPRQDGNDHGYMAQHDVAVSTDGSNWETVAAGTWYGGDKTLKYANFETRTVRYVRVRATSEANGGPWTSLAELKAYAAKTGPAPYAGLGKWGATIDFPTVPVAAAVDPVSGKVLVWSSYTYDNYLGSTQDRVFTSLWDPATGAVTPKLVDDTDHDMFCPGISIDGAGQMVVTGGNSASKTTLYDFASGAWLPGPDMTVARGYQASATLSDGRVFTIGGCWSGGWFDKNGEVYDPRARTWTGLPQALVRPMLTADAQGIYRADNHAWLFGWRNGSVFQAGPSTAMNWYATAGNGSVSPAGQRRSDRGADADAMNGNAVMFDALAGRILAFGGAPSYQDSQASAAAHLITIGDPGKPADVRFASNGLWSPRAFHTSAVLPDGTVFITGGQSYAVPFSDETPQLTPELYDPAADAFYKQQPNSIVRVYHSVALLLPDATVLSAGGGLCGDCNTNHFDGQVFTPQYLLTKDGQPAVRPVIRSATLSGRTVAIETDSSVASASLIRFGTATHTVNTDQRRVPLTLVRVGTNRYTAEVPADTGVVLPGYYMLFVMNEKGVPSVSKTLNFLV

SEQ ID NO: 67 - protein, 669 aa, Metarhizium rileyi RCEF 4871
MKLTTETCLLAALFAGQVSGLVSKEHYREKSTFSKLFAAPPIGNELNRAGWKVTCDSAEPGSECGKAIDGSNETFWHTSYDGSNLPHQIVVDFGSRHNINGISALPRQDGSDHGFIAQHDVAVSTDGRNWETVAGGTWHGGDSMLKYANFETRSARYVRVRATSEANGNAWTSLAELKAYEAKSGPTAYRRTGKWGPTINFPTVPVAATVDPGSGRVIVWSSYTYDNYLGSSQDRVFTSVWDPATGVVTPKLVDNTDHDMFCPGISIDGTGSMVVTGGNSASKTTLYDFASESWIPGPDMNFPRGYQASATLSDGRVFTIGGCWSGGWFEKNGEVYDPKARAWTNLTGARVRPMLTADAQGIYRADNHGWLFGWKGGSVFQAGPSTAMNWYTTSGDGGVSPAGQRESERGADGDSMNGNAVMYDATQGKILAVGGAPSYQDSPATGHAHLITIGDPGSEARVRFAGNGMSAARAFHNSVVLPDGTVFITGGQSYAVPFSDDTPDLTPELYDPATDAFTQQQPNSIVRVYHSVALLLPDARVLSAGGGLCGDCTTNHFDAQVFTPQYLLTRAGQPASRPAIRSADVRDGNIAIVTDSAVASASLVRFGTATHTVNTDQRRIPLRLVRSGTNRYTANVSLDPGILLPGYYMLFVLNANGVPSVSKTLKFLV

SEQ ID NO: 68 - protein, 676 aa, Metarhizium brunneum ARSEF 3297
MKLTTETVLLGALFAGQAAGGLVPSPLTGKQHYHENSTFSKLFAAPPIATGELDRAGWKVTCDSFEPGNECIKAIDGNNDTFWHTKFEGSNVPHQIVVDFGSMHNINGISALPRQDGNNHGYIAQHDVAVSTDGSNWETVAAGTWYGGDKLLKYANFETRTARYVRLRATSEVSGAPWTSVAELKAYAAKSGPAAYGGVGKWGATIDFPTVPVAAAVDPVSGKVLVWSSYTYDNYLGSTQDRVFTSLWDPATGAVTPKLVDDTDHDMFCPGISIDGTGQMVVTGGNSASKTTLYDFASGAWLPGPDMTVARGYQASATLSDGRVFTIGGCWSGGWFDKNGEVYDPRARAWSGLPGALVRPMLTADAQGIFRADNHAWLFGWRNGSVFQAGPSTAMHWYYTAGNGSVAPAGDRRSDRGTDPDAMNGNAVMFDARAGRILSFGGSPSYQNSQASAAAHLITIGDPGKPADVRFASNGLWSPRAFHTSAVLPDGTVFITGGQSYAVPFSDETPQLTPELYDPVADAFYKQQPNSIVRVYHSVALLLPDATVLSAGGGLCGDCNTNHFDGQVFTPQYLLTKDGQPAVRPVIRSATLSGRTVTIETDSSVASASLIRFGTATHTVNTDQRRVPLTLVRAGDNRYTAEVPADTGVVLPGYYMLFVMNDKGVPSVSKTLNFLV

SEQ ID NO: 69 - protein, 677 aa, Metarhizium anisopliae ARSEF 549
MKLTTETVLLGALFAGQAAGGLVPPSPLTGKQHYHENSTFSKLFAAPPIATGELDRAGWKVTCDSFEPGNECSLAIDGNNDTFWHTKFEGSNVPHQIVVDFGATHDINGISALPRQDGNNHGYIAAHEVAVSTDGSNWETVAAGTWYGGDKLLKYANFETRTARYVRLRATSEVSGAAWTSVAELKAYAAKSGPAAYGGAGKWGATIDFPTVPVAAAVDPVSGKVLVWSSYTYDNYLGSTQDRVFTSLWDPATGSVTPKLVDDTDHDMFCPGISIDGTGQMVVTGGNSASKTTLYDFASGAWLPGPDMTVARGYQASATLSDGRVFTIGGCWSGGWFDKNGEVYDPRARAWSGLPGALVRPMLTADAQGLFRADNHAWLFGWRNGSVFQAGPSTAMHWYYTAGNGSVAPAGDRRSDRGTDPDAMNGNAVMFDAPAGRILSFGGSPSYQNSDASAAAHLITIGDPGKPADVRFASNGLWSPRAFHTSAVLPDGTVFITGGQSYAVPFSDETPQLTPELYDPVADTFYKQQPNSIVRVYHSVALLLPDATVLSAGGGLCGDCNTNHFDGQVFTPQYLLTKDGQPAVRPVIRSATLSGRTVAIETDSSVASASLIRFGTATHTVNTDQRRVPLTLVRAGDNRYTAEVPADPGVVLPGYYMLFVMNDKGVPSVSKTLNFLV

SEQ ID NO: 70 - protein, 680 aa, Moelleriella libera RCEF 2490
MSRFLLLLALVAVASSAAVEQQQQQQQQPRAIKILEHYRESSTFTKLFAAAPIGNEIPKNAYTVTCDSYQPGNECALAIDGNNNTFWHTAFSGANLPHQIVVDLGATRNINGLSALPRQDGNNHGFIAQHEVAVSTDNRNWEVVASGTWYGGDSTTKFANFETRSIRYIRLKALSEAYGNQWTSVAEVKVYEAKTGPAAFAGTGKWGPTINFPTIPVAGAVDPLTGQVLIWSSYTYDNYLGSSQDRVFTSIWDPSTGVVTPKLVDNTNHDMFCPGISIDGAGRMVITGGNSAQKTTVYQFGSQTWIPGPDMNTQRGYQASATLSDGRVFTIGGCWSGGWFEKNGEVYDPKASTWTSLPGALVHPMLTNDAQGIYRADNHAWLFGWKNGSVFQAGPSTAMNWYTTSGNGSVAPAGNRTSSRGDAPDAMTGNAVMYDAVNGKILSFGGSPSYQDSSATTNAHIITIGSPGAPAQSRFASNGLWSPRAFHTSVILPDGKTFITGGQTYAVPFSDDNPDLTPEMYDPVADSFVQQQANTIIRVYHSISLLLPDGRVFNAGGGLCGDCTTNHFDGQIFTPQYLLTRTGQLAARPAINSVSLSGRRLTINTNSAITSAALMRYGSATHTVNTDQRRIPLKLTTTGTNRYTADAPSDPGILLPGYYMLFVLNSQGVPSVAKTVNFLV

SEQ ID NO: 71 - protein, 672 aa, Pochonia chlamydosporia 170
MKLASQTLLAIVSLSQAAAINSEITTKQHYRENSTFAKLSAAAPIGNEINHAGWKVTCDSYQPGNECIKAIDGEGKTFWHTAFDNSNLPHQIVVDLGTTHNVNGLSALPRQDGNNHGYIAQHDVSVSIDNENWETVARGNWYAGDSTQKFANFETKLIRYIRLRAMSEAHGHQWTSLAELKVYEAKGGPAAYSGNGKWGPTIDFPTVPVAGTVDPFTGKVIIWSSYKYDNYAGTSQDRVFTSIWDPATGIVTSKLVDDTDHDMFCPGISIDGTGKMVVTGGNSARKTTLYDYQKRTWIPGPDMNLPRGYQSSTTLSDGRIFTVGGCWSGGWLEKNGEVYDPKAKAWTNLSSALVRPMLTHDSQGIYRADNHGWLFGWKSGSVFQAGPSTSMNWYFTAGNGRVASAGIRKTIKGVDPDSMSGNAVMYDATNGKILTFGGSPSYQDSNATGHAHLITIGNPGSQAQVQVAGNGMWSPRAFHTSVVLPDGKTFITGGQSYAVPFSDDTAQLTPELYDPNTDTFYQQQSNTIIRVYHSMSLLLSDGRVFNAGGGLCGDCKTNHFDGQVFTPQYLLTNNGQPATRPVITSVIQSGRRITITTDSAVSTASLIRFGTATHTVNTDQRRIPLPLNTSGTNQYVADAPVDPGILLPGYYMLFVMNANGVPSVSKTLTFLV

SEQ ID NO: 72 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 73 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 74 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 75 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIADRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRIKLPNGARVEYEMQWGHIGWSVPAAFGYAVGAPERRNILMVGDGSFQLTAQEVAQMVRLKPPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 76 - protein, 568 aa, Zymomonas mobilis subsp. Mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 77 - protein, 568 aa, Zymomonas mobilis subsp. mobilis NRRL B-12526
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGVRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 78 - protein, 577 aa, Aspergillus parasiticus
MEGETLPLAQYLFKRLLQLGVDSIFGVPGDYNLTLLDHVVPSGLKWVGNCNELNAGYAADGYSRIKDIGAVVTTFGVGELSAINAIAGAYAEKAPVVHIVGTPMRASQESRALIHHTFNDGDYQRFDAIQEHVTVAQVSLSDHRTAPSEIDRILLQCLLHSRPVRIAIPVDMVPVLVPVAGLSSKIQIPPAVRQPQAEEAALNAVLKRIYSSKKPMILVDGETRSFGMLQRVNHFIQTIGWPTFTSGFGKGLVDETLPNVYGVCTLHQKAFVDSCDLVLVFGPHFSNTNSYNYFLKPADEKSVLFSPNSIQVNKDVFRDLPVGYFIEQLTQQLDISRIPTHKHDLVHPSLRTLPEVSPTDLVTQTGGFWKRFSPFLRTGDIILGETGTPGYGVNDFILPPQTRLFKPATWLSIGYMLPAALGASHAQRDLVASDQYHSLSNPRTILFIGDGSFQMTVQELSTIIHQKLNVIIFLINNDGYTIERCIHGRNQAYNDVAPWRYLKAAEFFGADQDGEYKASTWEVRTWADLDRVLNDSQLADGKGLRMVEVFMERLDAPDVLMGLLNNQVLRENAQSRL

SEQ ID NO: 79 - protein, 563 aa, FLS
MAMITGGELVVRTLIKAGVEHLFGLHGIHIDTIFQACLDHDVPIIDTRHEAAAGHAAEGYARAGAKLGVALVTAGGGFTNAVTPIANARTDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLNTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTLHFQQLAVGPNRVTGTRLENGSYHGVAAAFGADGYHVDSVESFSAALAQALAHNRPACINVAVALDPIPPEELILIGMDPFA

SEQ ID NO: 80 - protein, 563 aa, FLS L482S
MAMITGGELVVRTLIKAGVEHLFGLHGIHIDTIFQACLDHDVPIIDTRHEAAAGHAAEGYARAGAKLGVALVTAGGGFTNAVTPIANARTDRTPVLFLTGSGALRDDETNTLQAGIDQVAMAAPITKWAHRVMATEHIPRLVMQAIRAALSAPRGPVLLDLPWDILMNQIDEDSVIIPDLVLSAHGAHPDPADLDQALALLRKAERPVIVLGSEASRTARKTALSAFVAATGVPVFADYEGLSMLSGLPDAMRGGLVQNLYSFAKADAAPDLVLMLGARFGLNTGHGSGQLIPHSAQVIQVDPDACELGRLQGIALGIVADVGGTIEALAQATAQDAAWPDRGDWCAKVTDLAQERYASIAAKSSSEHALHPFHASQVIAKHVDAGVTVVADGGLTYLWLSEVMSRVKPGGFLCHGYLNSMGVGFGTALGAQVADLEAGRRTILVTGDGSVGYSIGEFDTLVRKQLPLIVIIMNNQSWGWTSHFQQLAVGPNRVTGTRLENGSYHGVAAAFGADGYHVDSVESFSAALAQALAHNRPACINVAVALDPIPPEELILIGMDPFA

SEQ ID NO: 81 - protein, 941 aa, Vibrio vulnificus
MHNGVMKAWLESSHLAGANATYVEDLYELYLSDPDLVSEEWKRVFEGLPKPTKEVAEQPHSRVRDYFRRLAQETKHYSVQVSDPDVDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGAKRFSLEGGDAMIPMMKELIRHAGRSGMREVVIGMAHRGRLNMLVNVLGKKPQDLFDEFAGKHGESWGTGDVKYHQGFSADFATPGGDVHLALAFNPSHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 82 - protein, 857 aa, Vibrio vulnificus
DVDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGAKRFSLEGGDAMIPMMKELIRHAGRSGMREVVIGMAHRGRLNMLVNVLGKKPQDLFDEFAGKHGESWGTGDVKYHQGFSADFATPGGDVHLALAFNPSHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 83 - protein, 857 aa, Vibrio vulnificus
DVDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGAKRFSLEGGDAMIPMMKELIRHAGRSGMREVVIGMAHRGRLNMLVNVLGKKPQDLFDEFAGKHGESWGTGDVKYHQGFSADFATPGGDVHLALAFNPNHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 84 - protein, 857 aa, Vibrio vulnificus
DVDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGALRFSLEGGDAMIPMMKELIRHAGRSGMREVVIGMAHRGRLNMLVNVLGKKPQDLFDEFAGKHGESWGTGDVKYHQGFSADFATPGGDVHLALAFNPSHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 85 - protein, 857 aa, Vibrio vulnificus
DVDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGALRFSLEGGDAMIPMMKELIRHAGRSGMREVVIGMAHRGRLNMLVNVLGKKPQDLFDEFAGKHGESWGTGDVKYHQGFSADFATPGGDVHLALAFNPNHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 86 - protein, 855 aa, Vibrio vulnificus
VDAKQVKVLQLINAYRFRGHEAAELDPLGLWQRPTVAELDPAFHNLTEDDFEETFNVGSFAVGQETMPLKDIYTALKKTYCGSIGAEYMHMTDTEQKRWIQQRLESVVGQPSFDKDEKRTFLAELTAAEGLERYLGAKFPGAKRFSLEGGDAMIPMMKELIRHAGRSGMREVVIGMAHRGRLNMLVNVLGKKPQDLFDEFAGKHGESWGTGDGGGQGFSADFATPGGDVHLALAFNPSHLEIVNPVVMGSVRARQDRLGDDDGSKVLPITIHGDSAIAGQGVVAETFNMSQARGFCVGGTVRVVVNNQVGFTTSNPRDTRSTMYCTDIAKMVQAPIFHVNADDPEAVAFVTRIALDYRNEFKRDVVIDLVCYRRHGHNEADEPNATQPLMYQKIKKHPTPRKLYADVLIDRNECDIETATQMVNEYRDALDHGEVVVKEWRPMALHSVDWSPYLGHEWDTPWSNTYDKQRLVELGKRLCQYPESHTLHSRVSKLYNDRTAMTNGEKELDWGMAETLAYATLVDDGKRIRISGQDSGRGTFFHRHAVLHNQNDASTYVPLANIHDKQGPFEVFDSVLSEEAVLAFEYGYATAEPSGLTLWEAQFGDFANGAQVVIDQFISSGEQKWARLCGLTMLLPHGYEGQGPEHSSARLERYLQLCAEQNMQVVVPSTPAQVYHMIRRQVVRPMRRPLIVMSPKSLLRHPLCTSSLDDLANGTFMPAIPEIDELDPAKVKRVVFCSGKVYFDLLEQRRNNEQDDVAIVRIEQLYPFPMDDVKAAIAPYVNVEDFVWCQEEPQNQGAWYCSQHNFRAAIPAGTELKYAGRPASASPAVGYMSVHLKQQKALIDDALNVNEKTSD

SEQ ID NO: 87 - protein, 683 aa, Syncephalastrum racemosum
MTLPTTIHPLDPLSPEEIRHVSEIIRKQRAADETTYIFNSIALREPPKEQILAHFGWTDGPKPVQIDRQAFAVLIDRPSGLVHEIIVSITTASIVSWETKEGVQPTLHVQEMLEAEQVMLKDERVIEECRKLGIEDMSMVFADTWGVGWHKTKGKRLMQALMYMRTSPDDNQYAHPLDFTPLYDVNEQKVIDVLVAKRRNSKFERPVIPRADRQFLPEHLGEENLRKDIKPIEITQPQGVSFQIRGHEIDWQKWNLHVGFNYREGLVINNVSYKDMDGTVRPMFYRVSLAEMVVPYANPYEPYNHKMAFDVGEYGLGNLTNSLELGCDCVGSIFYMDGVCSDIKGDAWVIPNAICIHEEDTGLLFKHTDFRNNKAHSARSRRLVISHIVTAANYDYGLYYYFYQDGTFQYEVKATGELNTHVLAEDEDPAPYGTIVAPQVDAQHHQHLFSMRIDPMVDGPTNSVAQVDVVASDLPVGHPDNAVGNAFSPVTTIYADTDEARARANGETSRYWKIINETRIHPYTKEPVGFKLMCPNTPPMLPKPGSIAYERAVFASNTVWVTPYDAEQLFPGGFYCYQSDPADRLGLPEWTREKKDVKNKDIVLWLTFGLTHIPRVEDFPIMPVETCGFMLKPCNFFLANPGIDIPASDRHSSKSAYAPAVANGEYGITNGTTNGSSCCSKGH

SEQ ID NO: 88 - protein, 638 aa, Arthrobacter sp.
MTETTAVNTDFTLLSEQEIATAKQLLVENQLFTGSTRIAYMGLEDPSEQNADRLVRVMLMDKARNEPKDVVLDLTRGEVASVIELDPAAVGQLPVLVEEFEMVEAILAEDAQWAAALEKRGLAVDQVRVAPLSAGVFYPESGRRILRGLAFRQDFADSAWAHPIDGLVVYIDTIEQKVDQLLDFGIVDVPATHGNYTDPELTGPIRTTQKPIEITQPEGASFTVTEGNHVEWEKWSLDIGFDMREGLVLYNIGFDDKGERRRILDRASIAEMVVPYGDPSPVRSWQNYFDTGEYLVGRFANSLELGCDCLGEIHYISPVITDADGNAQTITNGICMHEEDSSILAKHSDDWSGVKYTRRNRRLVISFFTTVGNYDYGFYWYLYLDGTIEFEAKATGIVFTSAMRDERFASEMAPGLGAPFHQHLFGARLDFALDGGPSRVIEEEAVRLPISEDNPRGNAFTRSQTVLATEKQAVRDANQAAGRTWVVTNPEKKNYLGKPVGFKLLPQGLPTLLAAEGSSVHRRAEFASKALWVTQRDAAHRYPTGDFVNQNPGVDGIGSWIEDDKSIDGEKISLWHTFALTHFPRTEDWPIMPVDTVGFKIRPEGFFDRSPVLDVPEPAKHGCCATTTEADGCCGSNG

SEQ ID NO: 89 - protein, 472 aa, Bacillus megaterium (strain ATCC 12872 / QMB1551)
MLQKWITSIGLDIGTSTTKLIVSKLLIANQQNQFTLPGCQIIDRRVTYASSIYTTPMLNDVEIDVQRLTVLLEQEYKNAGISLDQVEAGAVIITGETARKQNAESIVHYLAEHAGGFVVATAGADLEGILAAKGSGAIERSAETNAVIANIDVGGGTANIALCQNGKVIETFTLHVGGRLIRLNEDGLVTYVSSHLTEFLKNNSLTLREGEKATFEKLSSICQLLAEETVNYVKALNQNSSLLVSPHTRSSTPPETIMVSGGVGAMMEKQKPKTVKEVAIHGDVGPLLADHFQSIQVSQAAETTRATVIGAGMQNTEVSGSTVYIKSKKLPLKNIPIIEIPVQPEEEWNPQLFQERARASCMQASTIFSAEDPPVAIALSHFPYCSYKMLQELAKVISAEFIACFKRAKCLVVLCEQDIAKALGQALAKQRKELEIICLDQIDFTHGDYIDLGLPVAGEAISVSVKTLAFSS

SEQ ID NO: 90 - protein, 472 aa, Bacillus megaterium (A0A380XUP7)
MLQKWITSIGLDIGTSTTKLIVSKLLIANQQNQFTLPGCQIIDRQVTYASSIYTTPMLNEVEIDVQRLTVLLEQEYKNAEISLDQVEAGAVIITGETARKQNAETIVHYLAEHAGDFVVATAGADLEGILAAKGSGAIQHSAETNAVIANIDVGGGTANIALCQNGKVIETFTLHVGGRLIRLNSDGFVTYVSPYLTEFLKNNSLILREGEKATFEKLSSICQLLAEETVNYVRALIQTSSLLVSPHTRSSIQPETIMISGGVGAMMEKQKPKTVREVAVHGDIGPLLAYQFQSIHVSQAAETTRATVIGAGMQNTEVSGSTVYIKSKKLPLKNIPIIEIPVQPEEEWNTQLFQERARASCMQASTIFSAEDPPVAIALSHFPYCSYKMLQELAKVISAEFGACFKGAKCLVVLCEQDIAKALGQALAKQKKELEIICLDQIDFTHGDYIDLGLPVAGEAISVSVKTLAFSS

SEQ ID NO: 91 - protein, 438 aa, Arabidopsis thaliana
MASSSLCHRYFNKITVTPFFNTKKLHHYSPRRISLRVNRRSFSISATMSSSTKKVLIPVAHGTEPFEAVVMIDVLRRGGADVTVASVENQVGVDACHGIKMVADTLLSDITDSVFDLIMLPGGLPGGETLKNCKPLEKMVKKQDTDGRLNAAICCAPALAFGTWGLLEGKKATCYPVFMEKLAACATAVESRVEIDGKIVTSRGPGTTMEFSVTLVEQLLGKEKAVEVSGPLVMRPNPGDEYTITELNQVSWSFEGTPQILVPIADGSEEMEAVAIIDVLKRAKANVVVAALGNSLEVVASRKVKLVADVLLDEAEKNSYDLIVLPGGLGGAEAFASSEKLVNMLKKQAESNKPYGAICASPALVFEPHGLLKGKKATAFPAMCSKLTDQSHIEHRVLVDGNLITSRGPGTSLEFALAIVEKFYGREKGLQLSKATLV

SEQ ID NO: 92 - protein, 388 aa, Arabidopsis thaliana
MANSRTVLILCGDYMEDYEVMVPFQALQAFGITVHTVCPGKKAGDSCPTAVHDFCGHQTYFESRGHNFTLNATFDEVDLSKYDGLVIPGGRAPEYLALTASVVELVKEFSRSGKPIASICHGQLILAAADTVNGRKCTAYATVGPSLVAAGAKWVEPITPDVCVVDGSLITAATYEGHPEFIQLFVKALGGKITGANKRILFLCGDYMEDYEVKVPFQSLQALGCQVDAVCPEKKAGDRCPTAIHDFEGDQTYSEKPGHTFALTTNFDDLVSSSYDALVIPGGRAPEYLALNEHVLNIVKEFMNSEKPVASICHGQQILAAAGVLKGRKCTAYPAVKLNVVLGGGTWLEPDPIDRCFTDGNLVTGAAWPGHPEFVSQLMALLGIQVSF

SEQ ID NO: 93 - protein, 186 aa, Caenorhabditis elegans
MAAQKSALILLPPEDAEEIEVIVTGDVLVRGGLQVLYAGSSTEPVKCAKGARIVPDVALKDVKNKTFDIIIIPGGPGCSKLAECPVIGELLKTQVKSGGLIGAICAGPTVLLAHGIVAERVTCHYTVKDKMTEGGYKYLDDNVVISDRVITSKGPGTAFEFALKIVETLEGPEKTNSLLKPLCLAK

SEQ ID NO: 94 - protein, 236 aa, Candida albicans
MVKVLLALTSYNETFYSDGKKTGVFVVEALHPFEVFRKKGYEIQLASETGTFGWDDHSVVPDFLNGEDKEIFDNVNSEFNVALKNLKKASDLDPNDYDIFFGSAGHGTLFDYPNAKDLQKIATTVYDKGGVVSAVCHGPAIFENLNDPKTGEPLIKGKKITGFTDIGEDILGVTDIMKKGNLLTIKQVAEKEGATYIEPEGPWDNFTVTDGRIVTGVNPQSAVKTAEDVIAAFECN

SEQ ID NO: 95 - protein, 217 aa, Drosophila melanogaster
MLSVLRKSFPNGVTHAHRVIRCKSNQDKCAKNALIILAPGAEEMEFTISADVLRRGKILVTVAGLHDCEPVKCSRSVVIVPDTSLEEAVTRGDYDVVVLPGGLAGNKALMNSSAVGDVLRCQESKGGLIAAICAAPTALAKHGIGKGKSITSHPDMKPQLKELYCYIDDKTVVQDGNIITSRGPGTTFDFALKITEQLVGAEVAKEVAKAMLWTYKP

SEQ ID NO: 96 - protein, 187 aa, Drosophila melanogaster
MSKSALVILAPGAEEMEFIIAADVLRRAGIKVTVAGLNGGEAVKCSRDVQILPDTSLAQVASDKFDVVVLPGGLGGSNAMGESSLVGDLLRSQESGGGLIAAICAAPTVLAKHGVASGKSLTSYPSMKPQLVNNYSYVDDKTVVKDGNLITSRGPGTAYEFALKIAEELAGKEKVQEVAKGLLVAYN

SEQ ID NO: 97 - protein, 283 aa, Escherichia coli
MTVQTSKNPQVDIAEDNAFFPSEYSLSQYTSPVSDLDGVDYPKPYRGKHKILVIAADERYLPTDNGKLFSTGNHPIETLLPLYHLHAAGFEFEVATISGLMTKFEYWAMPQKDEKVMPFFEQHKSLFRNPKKLADVVASLNADSEYAAIFVPGGHGALIGLPESQDVAAALQWAIKNDRFVISLCHGPAAFLALRHGDNPLNGYSICAFPDAADKQTPEIGYMPGHLTWYFGEELKKMGMNIINDDITGRVHKDRKLLTGDSPFAANALGKLAAQEMLAAYAG

SEQ ID NO: 98 - protein, 291 aa, Escherichia coli
MTVQTSKNPQVDIAEDNAFFPSEYSLSQYTSPVSDLDGVDYPKPYRGKHKILVIAADERYLPTDNGKLFSTGNHPIETLLPLYHLHAAGFEFEVATISGLMTKFEYWAMPHKDEKVMPFFEQHKSLFRNPKKLADVVASLNADSEYAAIFVPGGHGALIGLPESQDVAAALQWAIKNDRFVISLCHGPAAFLALRHGDNPLNGYSICAFPDAADKQTPEIGYMPGHLTWYFGEELKKMGMNIINDDITGRVHKDRKLLTGDSPFAANALGKLAAQEMLAAYAGLEHHHHHH

SEQ ID NO: 99 - protein, 196 aa, Escherichia coli
MSASALVCLAPGSEETEAVTTIDLLIRGGIKVTTASVASDGNLAITCSRGVKLLADAPLVEVADGEYDVIVLPGGIKGAECFRDSTLLVETVKQFHRSGRIVAAICAAPATVLVPHDIFPIGNMTGFPTLKDKIPAEQWQDKRVVWDARVKLLTSQGPGTAIDFGLKIIDLLVGREKAHEVASQLVMAAGIYNYYE

SEQ ID NO: 100 - protein, 172 aa, Escherichia coli
MSKKIAVLITDEFEDSEFTSPADEFRKAGHEVITIEKQAGKTVKGKKGEASVTIDKSIDEVTPAEFDALLLPGGHSPDYLRGDNRFVTFTRDFVNSGKPVFAICHGPQLLISADVIRGRKLTAVKPIIIDVKNAGAEFYDQEVVVDKDQLVTSRTPDDLPAFNREALRLLGA

SEQ ID NO: 101 - protein, 329 aa, Glycine max
MAPKKVLLLCGDFMEDYEAMVPFQALQAFGLAVDAVCPGKKSGDVCRTAVHVLAGAQTYSETVGHNFSLNATFDEVDAASYDGLWVPGGRAPEYLAHVPGVVELVTKFVSLGKQIASICHGQLILAAAGVVKGRTCTAFPPVKPVLVAAGAHWVEPDTEAATVVDGDLITAATYEGHPELIRHFVKALGGKISGFDKKILFICGDYMEDYEVKVPFQSLQALGCHVDAVCPSKKAGDTCPTAVHDFEGDQTYSEKPGHTFALTATFDDVDPSGYDALVIPGGRAPEYLALNESVIALVKYFFENKKPVASICHGQQILSAAGVLKVLLL

SEQ ID NO: 102 - protein, 169 aa, Homo sapiens
MASKRALVILAKGAEEMETVIPVDVMRRAGIKVTVAGLAGKDPVQCSRDVVICPDASLEDAKKESAAVKEILKEQENRKGLIAAICAGPTALLAHEIGFGSKVTTHPLAKDKMMNGGHYTYSENRVEKDGLILTSRGPGTSFEFALAIVEALNGKEVAAQVKAPLVLKD

SEQ ID NO: 103 - protein, 135 aa, Mus musculus
MASKRALVILAKGAEEMETVIPVDVMRRAGIKVTVAGLAGKDPVQCSRDVMICPDTSLEDAKTQGPYDVVVLPGGNLGAQNLSESPMVKEILKEQESRKGLIAAICAGPTALLAHEVGFGCKVTTHPLAKDKMMN

SEQ ID NO: 104 - protein, 454 aa, Oryza sativa
MLPSSRYLLAPAPLPAMVVRPPPPHPPSRGTSPLARPPLCRAMARAAPSLSAAASTAASSSTTPAKKKVLLPIAMGTEEMEAVILAGVLRRAGADVTLASVEDGLEVEASRGSHIVADKRIAACADQVFDLVALPGGMPGSVRLRDSVILQRITVRQAEEKRLYGAICAAPAVVLMPWGLHKRKKITCHPSFIEDLPTFRTVESNVQVSGELTTSRGPGTAFQFALSFVEQLFGPCKAEDMDNTLLTKVDDNLERSIEVNEIEWSSDHNPHVLIPIANGSEEMEIIMLTDVLRRANVNVVLASVEKSTSIVGSQRMRIVADKCISDASALEYDLIILPGGPAGAERLHKSSVLKKLLKEQKQTGRMYGGICSSPVILQKQGLLQDKTVTAHPSIVNQLTCEVIDRSKVVIDGNLITGMGLGTVIDFSLAIIKKFFGHGRAKGVANGMVFEYPKS

SEQ ID NO: 105 - protein, 238 aa, Oryza sativa
MLPSSRYLLAPAPLPAMVVRPPPPHPPSRGTSPLARPPLCRAMARAAPSLSAAASTAASSSTTPAKKKVLLPIAMGTEEMEAVILAGVLRRAGADVTLASVEDGLEVEASRGSHIVADKRIAACADQVFDLVALPLTKVDDNLERSIEVNEIEWSSDHNPHVLIPIANGSEEMEIIMLTDVLRRANVNVVLASVEKSTSIVGSQRMRIVADKCISDASALEYDLIILPVSYFSFHIFI

SEQ ID NO: 106 - protein, 189 aa, Plasmodium falciparum 3D7
MSGKKTALVAVASGSEDVEYITVVDVLRRAGVHVTTASVEKSEQVCLQSKNVVLADTTISKVRNNIYDVLVIPGGMKGSNTISECSEFIDMLKEQKANNRLYAAICAAPETVLDRHSLIDDVEAVAYPSFERNFKHIGKGRVCVSKNCITSVGPGSAVEFGLKIVEHLLGRQVALSLASGFLLHPAVTF

SEQ ID NO: 107 - protein, 215 aa, Pseudomonas aeruginosa
MGRASTGKAAPGNGHGTFPAAARVGMNTPSDIGRHAMTQSLHGKVVAALVTDGFEQVELTGPKKALEDAGATVRILSDKAGEVRGWNHHQPAEAFRVDGTFEDASLDDYDALLLPGGVINSDQIRSLAKAQELAIRAEQASKPVAVICHGAWLLISAGLVQGRTLTSWPSLKDDINNAGGHWVDQEVAVDGKLVSSRKPEDIPAFNRRFIEILAG

SEQ ID NO: 108 - protein, 166 aa, Pyrococcus furiosus
MKILFLSANEFEDVELIYPYHRLKEEGHEVYIASFEKGVITGKHGYSVKVDLTFDEVNPDEFDALVLPGGRAPERVRLNEKAVEIARKMFTEGKPVATICHGPQILISAGVLKGRKGTSYIGIRDDMINAGVEWIDREVVVDGNWVSSRHPGDLYAWMREFVKLLK

SEQ ID NO: 109 - protein, 166 aa, Pyrococcus horikoshii
MKVLFLTANEFEDVELIYPYHRLKEEGHEVYIASFERGTITGKHGYSVKVDLTFDKVNPEEFDALVLPGGRAPERVRLNEKAVSIARKMFSEGKPVASICHGPQILISAGVLRGRKGTSYPGIKDDMINAGVEWVDAEVVVDGNWVSSRVPADLYAWMREFVKLLK

SEQ ID NO: 110 - protein, 245 aa, Saccharomyces cerevisiae
MAPKKVLLALTSYNDVFYSDGAKTGVFVVEALHPFNTFRKEGFEVDFVSETGKFGWDEHSLAKDFLNGQDETDFKNKDSDFNKTLAKIKTPKEVNADDYQIFFASAGHGTLFDYPKAKDLQDIASEIYANGGVVAAVCHGPAIFDGLTDKKTGRPLIEGKSITGFTDVGETILGVDSILKAKNLATVEDVAKKYGAKYLAPVGPWDDYSITDGRLVTGVNPASAHSTAVRSIVALKNLEHHHHHH

SEQ ID NO: 111 - protein, 237 aa, Saccharomyces cerevisiae
MAPKKVLLALTSYNDVFYSDGMKTGVFVVEALHPFNTFRKEGFEVDFVSETGKFGWDEHSLAKDFLNGQDETDFKNKDSDFNKTLAKIKTPKEVNADDYQIFMASAGHGTLFDYPKAKDLQDIASEIYANGGVVAAVCHGPAMFDGLTDKKTGRPLIEGKSITGFTDVGETIMGVDSILKAKNLATVEDVAKKYGAKYLAPVGPWDDYSITDGRLVTGVNPASAHSTAVRSIDALKN

SEQ ID NO: 112 - protein, 191 aa, Schizosaccharomyces pombe
MVKVCLFVADGTDEIEFSAPWGIFKRAEIPIDSVYVGENKDRLVKMSRDVEMYANRSYKEIPSADDFAKQYDIAIIPGGGLGAKTLSTTPFVQQVVKEFYKKPNKWIGMICAGTLTAKTSGLPNKQITGHPSVRGQLEEGGYKYLDQPVVLEENLITSQGPGTAMLFGLKLLEQVASKDKYNAVYKSLSMP

SEQ ID NO: 113 - protein, 547 aa, Lactococcus lactis
MYTVGDYLLDRLHELGIEEIFGVPGDYNLQFLDQIISREDMKWIGNANELNASYMADGYARTKKAAAFLTTFGVGELSAINGLAGSYAENLPVVEIVGSPTSKVQNDGKFVHHTLADGDFKHFMKMHEPVTAARTLLTAENATYEIDRVLSQLLKERKPVYINLPVDVAAAKAEKPALSLEKESSTTNTTEQVILSKIEESLKNAQKPVVIAGHEVISFGLEKTVTQFVSETKLPITTLNFGKSAVDESLPSFLGIYNGKLSEISLKNFVESADFILMLGVKLTDSSTGAFTHHLDENKMISLNIDEGIIFNKVVEDFDFRAVVSSLSELKGIEYEGQYIDKQYEEFIPSSAPLSQDRLWQAVESLTQSNETIVAEQGTSFFGASTIFLKSNSRFIGQPLWGSIGYTFPAALGSQIADKESRHLLFIGDGSLQLTVQELGLSIREKLNPICFIINNDGYTVEREIHGPTQSYNDIPMWNYSKLPETFGATEDRVVSKIVRTENEFVSVMKEAQADVNRMYWIELVLEKEDAPKLLKKMGKLFAEQNK

(19) United States
(12) Patent Application Publication
(10) Pub. No.: US 2023/0109331 A1
(43) Pub. Date: Apr. 6, 2023

(54) COMPOSITIONS, SYSTEMS AND METHODS FOR PRODUCTION OF VALUE-ADDED CHEMICALS

(71) Applicant: Solugen, Inc., Houston, TX (US)

(72) Inventors: Toni M. Lee, Missouri City, TX (US); Shuai Qian, Houston, TX (US); Brian F. Fisher, Houston, TX (US); Konrad V. Miller, Sugar Land, TX (US); Philipp Wiemann, Houston, TX (US); Gaurab Chakrabarti, Houston, TX (US); Sean Hunt, Houston, TX (US)

(73) Assignee: Solugen, Inc., Houston, TX (US)

(21) Appl. No.: 17/790,923

(22) PCT Filed: Jan. 6, 2021
(86) PCT No.: PCT/US2021/012351

§ 371 Date: Jul. 5, 2022

Related U.S. Application Data
(60) Provisional application No. 62/957,678, filed on Jan. 6, 2020.
(60) Provisional application No. 62/969,475, filed on Feb. 3, 2020.
(60) Provisional application No. 62/989,460, filed on Mar. 13, 2020.
(60) Provisional application No. 63/037,440, filed on Jun. 10, 2020.
(60) Provisional application No. 63/060,721, filed on Aug. 4, 2020.
(60) Provisional application No. 63/071,753, filed on Aug. 28, 2020.
(60) Provisional application No. 63/090,784, filed on Oct. 13, 2020.

(51) Int. Cl. C12P 13/00; C12P 7/56; C12P 7/20; C12P 7/24; C12P 7/28; C12N 9/04; C12N 9/06; C12N 9/88; C12N 9/08

(52) U.S. Cl. CPC C12P 13/00; C12P 7/56; C12P 13/001; C12P 7/20; C12P 7/24; C12P 7/28; C12N 9/0006; C12Y 101/03013; C12N 9/0022; C12N 9/88; C12Y 404/01005; C12Y 111/01006; C12N 9/0065

(57) ABSTRACT

A system for the production of high value chemicals includes (a) an input selected from the group consisting of ethylene glycol, glycerol, ethanol methanol or a combination thereof. In addition, the system includes (b) an oxidation biocatalyst including an alcohol oxidase, a copper radical oxidase, a glycerol oxidase, an alditol oxidase or a combination thereof. Further, the system includes (c) an oxidized intermediate. The system also includes (d) a finishing catalyst including a supported metal catalyst, a carboligating catalyst, an amine oxidase, a glyoxalase, an acid catalyst, a base catalyst, an isomerization catalyst or a combination thereof. Still further, the system includes (e) an output.

### CROSS-REFERENCE TO RELATED APPLICATIONS

The present application is a 35 U.S.C. § 371 U.S. National Stage Entry application of PCT/US2021/012351 filed Jan. 6, 2021 and entitled “Compositions, Systems, and Methods for Production of Value-Added Chemicals,” which claims priority to U.S. Provisional Patent Application No. 62/989,460 filed Mar. 13, 2020 and entitled “COMPOSITIONS AND METHODS FOR ACETALDEHYDE PRODUCTION FROM ETHANOL”; U.S. Provisional Patent Application No. 63/037,440 filed Jun. 10, 2020 and entitled “COMPOSITIONS AND METHODS FOR GYCOLIC ACID PRODUCTION”; U.S. Provisional Patent Application No. 63/060,721 filed Aug. 4, 2020 and entitled “COMPOSITIONS AND METHODS FOR DIHYDROXYACETONE PRODUCTION FROM GLYCEROL”; U.S. Provisional Patent Application No. 63/071,753 filed Aug. 28, 2020 and entitled “COMPOSITIONS AND METHODS FOR ETHANOLAMINE PRODUCTION FROM ETHYLENE GLYCOL”; U.S. Provisional Patent Application No. 63/090,784 filed Oct. 13, 2020 and entitled “COMPOSITIONS AND METHODS FOR PRODUCTION OF GLYCEROL FROM ETHYLENE GLYCOL”; U.S. Provisional Patent Application No. 62/969,475 filed Feb. 3, 2020 and entitled “IN VITRO ENZYMATIC FORMALDEHYDE PRODUCTION FROM METHANOL”; and U.S. Provisional Patent Application No. 62/957,678 filed Jan. 6, 2020 and entitled “IN VITRO LACTATE PRODUCTION FROM GLYCEROL”; each of which is incorporated by reference herein in its entirety for all purposes.

### STATEMENT REGARDING FEDERALLY SPONSORED RESEARCH OR DEVELOPMENT

Not applicable.

### TECHNICAL FIELD

The present disclosure relates generally to compositions, systems and methods for the production of value-added chemicals. More particularly, the present disclosure relates to chemoenzymatic systens and methods for the conversion of alcohols and polyols to higher value chemicals.

### BACKGROUND

Oxidation is a key reaction in organic synthesis and plays a significant role in the production of value-added chemicals. For example, mono- and polyalcohols (e.g., ethanol, and glycerol) are important platform molecules for industrial manufacturing of a large number of value-added products, including but not limited to aldehydes, ketones, ethers, and molecular hydrogen. Another essential and important step in the synthesis of many valuable products including polymers and pharmaceuticals is the oxidation of amines. Challenges in the production of these valuable products include the use of reagents and conditions that are harsh, costly, and environmentally unfriendly. Further, the reactions typically result in a mixture of products and impurities that require substantial processing to obtain the compound(s) of interest at an acceptable percentage purity. Thus, an ongoing need exists for systems, methods and compositions that produce high purity, value-added chemicals via oxidation.

### SUMMARY

A system for the production of high value chemicals, comprising (a) an input selected from the group consisting of ethylene glycol, glycerol, ethanol methanol and a combination thereof; (b) an oxidation biocatalyst comprising an alcohol oxidase, a copper radical oxidase, a glycerol oxidase, an alditol oxidase or a combination thereof; (c) an oxidized intermediate; (d) a finishing catalyst comprising a supported metal catalyst, a carboligating catalyst, an amine oxidase, a glyoxalase, an acid catalyst, a base catalyst, an isomerization catalyst or a combination thereof; and (e) an output.

A method for the production of high value chemicals, comprising (a) contacting an input selected from the group consisting of ethylene glycol, glycerol, ethanol methanol and a combination thereof with an oxidation biocatalyst comprising an alcohol oxidase, a copper radical oxidase, a glycerol oxidase, an alditol oxidase or a combination thereof to form an oxidized intermediate; (b) contacting the oxidized intermediate with a finishing catalyst comprising a supported metal catalyst, a carboligating catalyst, an amine oxidase, a glyoxalase, an acid catalyst, a base catalyst, an isomerization catalyst or a combination thereof to form an output.

### BRIEF DESCRIPTION OF DRAWINGS

For a detailed description of the aspects of the disclosed processes and systems, reference will now be made to the accompanying drawings in which:

FIG. 1 is a schematic depiction of a High Value Chemical Output System, termed “HVCOS”, of the present disclosure.

### DETAILED DESCRIPTION

The instant application contains a Sequence Listing which has been submitted electronically in ASCII format and is incorporated herein by reference in its entirety for all purposes. Said ASCII copy, created on Dec. 26, 2020 is named HVC_ST25.txt and is 591,000 bytes in size.

Disclosed herein are aspects of systems, methods and compositions for the conversion of alcohols, polyols, and amines to higher value chemicals (HVC). Also disclosed herein are aspects of processes for the conversion of alcohols, polyols, and amines to an oxidized intermediate which is subsequently converted to an output that may be further processed or used as a final product.

In one or more aspects, the compositions and methods disclosed herein result in a final product that is an HVC and may utilized without further processing. Alternatively, the compositions and methods disclosed herein result in a final product that is additionally processed to generate an HVC. For purposes of simplicity and further explanation, a final product of the methods and compositions disclosed herein, whether or not subjected to additional processing, is termed an “HVC.”

In an aspect, the production of an HVC is carried out according to a configuration having a first input to a first catalyst system, hereinafter referred to as the “oxidation biocatalyst,” to produce an intermediate. The intermediate may be subsequently used as an input (i.e., a second input) to a second catalyst system, hereinafter referred to as a “finishing catalyst,” to produce an output. In some aspects, the first input, the intermediate (i.e., second input), the output, or a combination thereof comprises a mixture of molecules or compounds.

In an aspect, the oxidation biocatalyst comprises a biocatalyst, which when contacted with the first input, under suitable conditions, oxidizes the first input to form one or more oxidized intermediates. The one or more oxidized intermediates, as produced or following processing, may be contacted with the finishing catalyst, under conditions suitable for the formation of an output. In one or more aspects, the output is a final product that may be used as produced or may be further processed to achieve some user and/or process goal.

It is contemplated that the oxidation biocatalyst, the finishing catalyst, or both may comprise one or more catalytic species, one or more cofactors, or both as desired to obtain some user and/or process desired characteristics such as reaction rate, catalyst stability, and the like. Configurations of the type disclosed herein are referred to as “high value chemical output systems” and are designated “HVCOS.”

It is contemplated that the systems, compositions, and methods disclosed herein may be utilized or readily adapted to the production of any number of HVCs. Consequently, while particular inputs, oxidation biocatalysts, oxidized intermediates, finishing catalysts, and outputs are described herein, it should be appreciated that these are exemplary and nonlimiting.

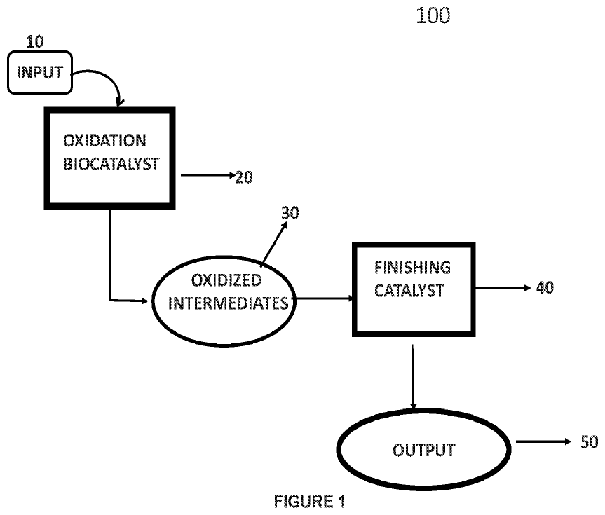

A schematic depiction of an aspect of a HVCOS 100 of the present disclosure is shown in FIG. 1. Referring to FIG. 1, a method of the present disclosure comprises introduction of a first input 10 to an oxidation biocatalyst 20. Herein, introduction of the first input 10 to the oxidation biocatalyst 20 involves contacting of the first input 10 with the oxidation biocatalyst 20 under conditions suitable for the formation of an oxidized intermediate 30. Such conditions will be described in more detail below. In an aspect, the first input 10 comprises ethylene glycol, glycerol, ethanol, methanol, or a combination thereof. In an aspect, the oxidation biocatalyst 20 comprises an alcohol oxidase, a copper radical oxidase, a glycerol oxidase, an alditol oxidase, or a combination thereof.

It is to be understood that while the oxidation biocatalyst may be specifically described in terms of the input being oxidized (e.g. oxidizes ethylene glycol, termed an ethylene glycol oxidase), the oxidation biocatalyst may catalyze oxidation of a range of substrates and/or a range of reactions in addition to those presently disclosed.

As depicted in FIG. 1, the oxidized intermediate 30 may be introduced as an input to the finishing catalyst 40. Herein introduction of the oxidized intermediate 30 to the finishing catalyst 40 involves contacting of the oxidized intermediate 30 with the finishing catalyst 40 under conditions suitable for the formation of an output 50. Such conditions will be described in more detail later herein. In an aspect, the finishing catalyst 40 comprises a supported metal catalyst, a carboligating catalyst, an amine oxidase, a glyoxalase, an acid catalyst, a base catalyst, an isomerization catalyst or a combination thereof. In some aspects, the oxidized intermediate 30 and output 50 are the same. In such aspects, the oxidized intermediate 30 is not contacted with a finishing catalyst 40.

In an aspect of the present disclosure, the output 50 is characterized by a percentage purity of from about 60% to about 95%, alternatively greater than about 70%, alternatively greater than about 80%, alternatively greater than about 90%, alternatively greater than about 95% or alternatively greater than about 99%. Herein, the percentage purity has its standard definition and is calculated by dividing the mass of the product of interest by the total mass of the product, and then multiplying this number by 100.

In an aspect, the oxidation biocatalyst is an alcohol oxidase (AOX, E.C. 1.1.3.13) or alcohol oxidase homolog. AOX is a ubiquitous flavin-dependent enzyme that oxidizes lower primary alcohols to aldehydes using oxygen as an oxidizing agent. An example is depicted in Reaction Scheme 1.

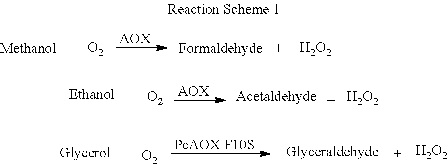

AOX may be sourced from methylotrophic yeast of the species Kloeckera, Torulopsis, Candida, Pichia, Hanseniaspora, and Metschnikowia. In an alternative aspect, the AOX is sourced from methanol-utilizing bacteria such as Methylococcus capsulatus, thermophilic soil fungi such as Thermoascus aurantiacus, and brown rot fungus such as Gloeophyllum trabeum. Alternatively, the AOX may be sourced from the white-rot basidiomycete Phanerochaete chrysosporium.

Methylotrophic yeasts are widely employed in fermentative processes for protein production and chemical synthesis. In many cases, these yeasts are used to generate proteins heterologously under control of the methanol-inducible AOX1 promoter. The endogenous AOX1 gene can be retained (Mut⁺ strains), deleted (Muts), or deleted along with that of the minor alcohol oxidase AOX2 (Mut⁻). Generally, higher protein titers are achieved in strains capable of utilizing methanol as a carbon source, while AOX genes may be deleted to improve protein titers in non-methanol induced processes.

In an aspect, the AOX is sourced from Mut⁺ cells generated as a byproduct of methylotrophic yeast fermentation. Cell density in these processes can reach a final level of from about 350 g/L to about 450 g/L wet cells. When grown in methanol, AOX can comprise 30% of soluble cellular protein, 20% of cell-free extracts, and 80% of cell volume. Alternatively, AOX sequences used in this process may be sourced from organisms other than methylotrophic yeasts.

In an aspect, the oxidation biocatalyst is an inherently stable form of an AOX from thermophilic organisms such as Candida methanosorbosa (Tₒₚₜ=45° C.), Ogataea thermomethanolica (Tₒₚₜ=50° C.), or Phanerochaete chrysosporium (Tₒₚₜ=50° C.). Potential sources of AOX suitable for use in the present disclosure are given in Table 1.

TABLE 1
Organism

| | |
|---|---|
| Achatina achatina | Achatina fulica |
| Arion ater | Aspergillus ochraceus |
| Aspergillus nidulans | Aspergillus terreus |
| Basidiomycota | Byssochlamys spectabilis |
| Candida boidinii | Candida cariosilignicola |
| Candida guilliermondii | Candida methanolovescens |
| Candida methanosorbosa | Candida sonorensis |
| Candida sithepensis | Candida sp. (in: |
| Candida succiphila | Candida tropicalis |
| Comamonas sp. | Hansenula polymorpha |
| Gloeophyllum trabeum | Helix aspersa |
| Kuraishia capsulata | Lachnellula arida |
| Lachnellula cervina | Lachnellula occidentalis |
| Lachnellula subtilissima | Lachnellula suecica |
| Lachnellula willkommii | Methylococcus capsulatus |
| Methylophilus methylotrophus | Ochrobactrum sp. |
| Ogataea glucozyma | Ogataea henricii |
| Ogataea methanolica | Ogataea minuta |
| Ogataea naganishii | Ogataea philodendri |
| Ogataea pignaliae | Ogataea pini |
| Ogataea siamensis | Ogataea trehalophila |
| Passalora fulva | Penicillium chrysogenum |
| Penicillium purpurascens | Phanerochaete |
| Pichia pastoris | Komagataella pastoris IFP |
| Phlebiopsis gigantea | Pichia putida |
| Thermoascus aurantiacus | Poria contigua CCA41016.1 |
| Radulodon casearius | Trametes cinnabarina |

An AOX for use in the present disclosure may be utilized in the oxidation of ethylene glycol and in such instances is termed an ethylene glycol oxidase or EgOX.

In an aspect, the oxidation biocatalyst is a member of the copper radical oxidase family. For example, and without limitation, a copper radical oxidase suitable for use in the present disclosure is galactose oxidase (GAO, EC 1.1. 3.9). GAO is one of the most extensively studied alcohol oxidases with respect to both mechanistic investigations and practical applications. Other members in the copper radical oxidase family may be suitable employed in the present disclosure. GAO is a copper-dependent alcohol oxidase that oxidizes galactose residues either as monosaccharides or glycoconjugates that contain galactose at the nonreducing end. GAO is a novel metallo-radical complex comprising a protein radical coordinated to a copper ion in the active site. The unusually stable protein radical is formed from the redox-active side chain of a cross-linked tyrosine residue (Tyr-Cys).

In an aspect, the GAO is a mutated GAO isolated from Fusarium graminearum GAO (FgGAO) or a native GAO homolog from Colletotrichum spinosum (CsAlcOX), both of which are capable of oxidizing ethylene glycol. The FgGAO sequence containing the M1 mutations and R330K, Q406T, and W290F, and the C383S mutation and the mutations Y405F Q406E was able to oxidize 50 mM ethylene glycol with a specific activity of about 18 U mL⁻¹ bacterial lysate and 3.9 U mg⁻¹ with purified protein. This mutant has been designated FgGAO-Mut1. The FgGAO-M-RQW-S mutant (M1 mutations plus R330K, Q406T, W290F, C383S) was also found to be active on ethylene glycol. CsAlcOX exhibited a specific activity for the oxidation of 50 mM ethylene glycol of about 0.6 U mL⁻¹. The FgGAO-M-RQW-S, FgGAO-Mut1, CsAlcOX may have any of SEQ ID NO.:1 to SEQ ID NO.:5. In an alternative aspect, the GAO has any of SEQ ID NO: 6

In an aspect, the oxidation biocatalyst is a glycerol oxidase (GlyOx). GlyOx (E.C. 1.1.3.64) catalyzes the oxidation of glycerol with the concomitant consumption of oxygen to form glyceraldehyde and hydrogen peroxide according to the following reaction:

CH₂OHCHOHCH₂OH+O₂→CHOCHOHCH₂OH+H₂O₂

The reaction proceeds in the absence of exogeneous cofactors. Natural glycerol oxidases containing copper-heme cofactors have been sourced from Botrytis allii, Aspergillus japonicus (AT 001 and AT 008), Aspergillus oryzae AT 105, Aspergillus parasiticus AT 462, Aspergillus flavus AT 853, Aspergillus tamarii AT 857, Aspergillus itaconicus AT 923, Aspergillus usamii AT 989, Neurospora crassa AT 003, Neurospora sitophila AT 045, Neurospora tetrasperma AT 053, and Penicffium sp. UT 1750.

In an aspect, the oxidation biocatalyst is an alditol oxidase (AldO, E.C. 1.1.3.41). AldO is a soluble monomeric flavoprotein with subunits of 45.1 kDa, each containing a covalently bound FAD cofactor. AldOs are FAD-bound polyol oxidases which are members of the vanillyl-alcohol oxidase (VAO) family that catalyze the regioselective terminal oxidation of sugars including xylitol, sorbitol, L-threitol, D-mannitol, and smaller sugars. When fed a glycerol substrate, the AldO from Streptomyces coelicolor A3 can catalyze conversion of the sugar to glyceraldehyde. The enzyme performs a second oxidation event to transform glyceraldehyde into D-(R)-glyceric acid as depicted in Reaction Scheme 2.

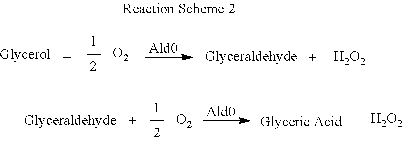

In an aspect, an AldO suitable for use in the present disclosure is a wild type enzyme, a functional fragment thereof or a functional variant thereof. In an aspect, the AldO is a mutant having one or more mutations selected from the group consisting of the mutation of residues R322, E290, and K375.

In an aspect, the finishing catalyst comprises a metal oxidation catalyst. In such aspects the metal oxidation catalyst is a supported transition-metal oxidation catalyst, alternatively a nanoparticle supported transition-metal oxidation catalyst. Hereinafter, these are collectively designated “TMC.” In an aspect, the support comprises carbon, silica, alumina, titania (TiO₂), zirconia (ZrO₂), a zeolite, or any combination thereof, which contains less than about 1.0 weight percent (wt. %), alternatively less than about 0.1 wt. %, or alternatively less than about 0.01 wt. % SiO₂ binders based on the total weight of the support.

Suitable support materials are predominantly mesoporous or macroporous, and substantially free from micropores. For example, the support may comprise less than about 20% micropores. In an aspect, the support is a porous nanoparticle support. As used herein, the term “micropore” refers to pores with diameter<2 nm, as measured by nitrogen adsorption and mercury porosimetry methods and as defined by IUPAC. As used herein, the term “mesopore” refers to pores with diameter from ca. 2 nm to ca. 50 nm, as measured by nitrogen adsorption and mercury porosimetry methods and as defined by IUPAC. As used herein, the term “macropore” refers to pores with diameters larger than 50 nm, as measured by nitrogen adsorption and mercury porosimetry methods and as defined by IUPAC.

In an aspect, the support comprises a mesoporous carbon extrudate having a mean pore diameter ranging from about 10 nm to about 100 nm and a surface area greater than about 20 m² g⁻¹ but less than about 300 m² g⁻¹. Supports suitable for use in the present disclosure may have any suitable shape. For example, the support may be shaped into 0.8-3 mm trilobes, quadralobes, or pellet extrudates. Such shaped supports enable the used of fixed trickle bed reactors to perform the final oxidation step under continuous flow.

In one or more aspects, the metal comprises a Group 8 metal (e.g., Re, Os, Ir, Pt, Ru, Rh, Pd, Ag), a 3d transition metal, an early transition metal, or combinations thereof. In an aspect, the TMC comprises gold, Au.

In an aspect, the TMC comprise platinum and gold and are heterogeneous, solid-phase TMCs. In such aspects, suitable catalyst supports include, without limitation, carbon, surface treated aluminas (such as passivated aluminas or coated aluminas), silicas, titanias, zirconias, zeolites, montmorillonites, and modifications, mixtures or combinations thereof. The catalyst support may be treated so as to promote the preferential deposition of platinum and gold on the outer surface of the support so as to create a shell type TMC. The platinum and gold-containing compounds that function as a TMC may be produced by any suitable methodology. For example, the platinum and gold-containing TMCs may be produced using deposition procedures such as incipient wetness, ion-exchange and deposition-precipitation.

In other aspects, the finishing catalyst is a TMC comprising metal phases that are monometallic or multimetallic combinations of Cu, Ag, Au, Ni, Pd, Pt, or Ir. The activity, selectivity, and stability of the active phases can be modulated with dopants of early 3d, 4d, and 5d transition metals, or heavy post transition metals such as Sn, Sb, and Bi. In some aspects, metals (e.g., Group 1 metals) are intercalated into the metal lattice to modulate catalyst properties. In an aspect, salt precursors of the active phases are deposited onto a support of the type disclosed herein using any suitable methodology. For example, deposition of the active phases may be carried out using techniques such as incipient wetness impregnation, bulk adsorption impregnation, or deposition precipitation.

In an aspect, the deposited salt precursor of the active phase is then converted to the active phase via Liquid Phase Reduction (LPR) with a suitable salt (e.g., formate salt) at temperatures of less than about 100° C. or via Gas Phase Reduction (GPR) at temperatures ranging from about 200° C. to about 500° C. or alternatively from about 200° C. to about 450° C. In an aspect, the finishing catalyst comprises gold, platinum or a combination thereof and calcination in air at temperatures of equal to or greater than about 150° C. is performed.

In an aspect, the amount of active phase loaded onto a support of the type disclosed herein is less than about 2.0 weight percent (wt. %), alternatively less than about 1.5 wt. % or alternatively less than about 1.0 wt. % based on the total weight of the TMC finishing catalyst. In an aspect, the amount of active phase loaded onto a support of the type disclosed herein is equal to or less than about 0.5 wt. % based on the total weight of the TMC finishing catalyst. In an aspect, the radial distribution of the active phase across the support is anisotropic where the active phase is substantially concentrated in a <500 μm annulus near the surface of the extrudate support in a “core-shell” configuration. A TMC finishing catalyst of the type disclosed herein may be characterized by a productivity for the conversion of aldehyde functionalities to carboxylic acids of equal to or greater than about 0.05 mol acid g⁻¹ active metal h⁻¹ or equal to or greater than about 0.1 mol acid g⁻¹ active metal h⁻¹ at selectivities from about 70% to about 90%, alternatively equal to or greater than about 70%, alternatively equal to or greater than about 80%, alternatively equal to or greater than about 85%, or alternatively equal to or greater than about 90%. In such aspects, the TMC finishing catalyst exhibits conversions of from about 60% to about 95%, alternatively equal to or greater than about 70%, alternatively equal to or greater than about 80%, or alternatively equal to or greater than about 90%. Such TMC finishing catalysts may display a steady state leaching amount of from about 1 ppb to about 100 ppb, alternatively less than about 100 ppb or alternatively less than about 90 ppb. In an aspect, a TMC finishing catalyst of the type disclosed herein may be utilized in a temperature range of from about 40° C. to about 120° C., alternatively form about 40° C. to about 110° C. or alternatively from about 50° C. to about 100° C. at pressures ranging from about 10 bar to about 100 bar, alternatively from about 20 bar to about 100 bar or alternatively from about 20 bar to about 90 bar.

In some aspects, the finishing catalyst is an isomerization catalyst. Any isomerization catalyst compatible with the other components of the HVCOS may be utilized. In some aspects, the isomerization catalyst comprises a zeolite.

In an aspect, the finishing catalyst comprises a carboligating catalyst. In such aspects, the carboligating catalyst comprises pyruvate decarboxylase (PDC) from prokaryote microorganisms, eukaryotic microorganisms or yeast, formolase, the E1 component of α-ketoglutarate dehydrogenase complex from SucA, the branched-chain alpha-keto acid decarboxylase, KdcA protein, of Lactococcus lactis, which decarboxylates a variety of branched and linear ketoacid substrates, B1 (thiamine) cofactor, or combinations thereof.

In an aspect, the carboligating catalyst is a pyruvate decarboxylase (PDC). Pyruvate decarboxylase (E.C. 4.1.1.1, also known as 2-oxo-acid decarboxylase, alpha-ketoacid decarboxylase, and pyruvic decarboxylase) is a homotetrameric enzyme that catalyzes the decarboxylation of pyruvic acid to produce acetaldehyde using magnesium and a thiamine pyrophosphate (TPP) cofactor. The enzyme is found in the cytoplasm of prokaryotes, and in the cytoplasm and mitochondria of eukaryotes. In yeast, this enzyme is a key player in the fermentation process that produces ethanol. A homolog of the yeast PDC is also present in filamentous fungi, e.g., Aspergillus spp., but less is known about its exact reaction mechanism.

Once regarded as irreversible due to CO₂ evolution in the biologically-relevant reaction, decarboxylase reactions have proved reversible. In an aspect, a PDC suitable for use in the present disclosure may be sourced from microbes with high specific activity on pyruvate including those sourced from Acetobacter pasteurianus PDC, Zymobacter palmae PDC (ZpPDC), Zymomonas mobilis PDC (ZmPDC), Saccharomyces cerevisiae PDC, or other sources mentioned herein.

In an aspect, the carboligating catalyst is a formolase. Formolase (FLS) is a computationally-designed enzyme derived from benzaldehyde lyase (BAL). Formolase contains the mutations mutant A28I, W89R, L90T, R188H, A394G, G419N, and A480W.

In an aspect, the carboligating catalyst is a KdcA from Lactococcus lactis. The natural substrate of KdcA decarboxylation is 3-methyl-2-oxobutanoic acid or α-ketoisovaleric acid to generate 2-methylbutanal. This enzyme has demonstrated the ability to carboligate carbon dioxide and 4-methylthio-2-oxobutanoate (MTOB) to produce L-methionine. The enzyme has also been shown to decarboxylate pyruvate with a specific activity of 3.67 U mg⁻¹. As such, it appears that KdcA is a promiscuous enzyme that can accept a wide range of branched and non-branched substrates for both decarboxylation and carboligation.

In an aspect, the carboligating catalyst comprises an immobilized vitamin B1 (or thiamine). For example, the carboligating catalyst can comprise thiamine immobilized to nanoparticles such as silica or maghemite-silica beads. In such aspects, a resonance-stabilized conjugate base of the thiazolium ion, thiamine, and the resonance stabilized carbanion (C) that it forms, are intermediats formed during the reaction. In an aspect, the carboligating catalyst may have any of SEQ ID NO:72 through SEQ ID NO: 86 or SEQ ID NO: 113.

In an aspect, the finishing catalyst is an amine oxidase (AMO, 1.4.3). AMOs can be grouped as either type I enzymes which contain a copper and topaquinone (TPQ or 2,4,5-trihydrophenylalanine quinone) in the active site or as type II enzymes which contain one FAD per subunit. AMOs suitable for use as a biocatalyst in the present disclosure include, without limitation, 1) monoamine oxidases (MAOs, EC 1.4.3.4), 2) primary-amine oxidases or amine oxidase (copper containing) (CAOs, EC 1.4.3.21), and 3) ethanolamine (EA) oxidases (EAOs, EC 1.4.3.8). In an aspect, an AMO suitable for use in the present disclosure has any of SEQ ID NO: 87 through SEQ ID NO:90.

In an aspect, the finishing catalyst is a monoamine oxidase (MAO). MAOs are mitochondrial outer-membrane type II AMOs that natively catalyze the reversible oxidative deamination of neurotransmitters and biogenic amines. MAOs act on primary amines, but to a lesser extent on secondary and tertiary amines. Unlike CAOs, MAOs cannot oxidize methylamine. In an aspect, an MAO suitable for use in the present disclosure is sourced from the bunch-flowered daffodil (Narcissus tazetta) that was found to use EA as a substrate with a Kₘ of 2.0×10⁻³ M and 50% the activity on the proposed native substrate n-propylamine.

In an aspect, the finishing catalyst is a copper-containing primary amine oxidase or copper-containing amine oxidase (CAO). CAOs are type I AMOs that reversibly oxidize primary monoamines but have little or no activity towards diamines. In the native forward reaction, catalysis proceeds via a Ping Pong Bi Bi reaction mechanism consisting of an oxidative half where molecular oxygen is reduced to hydrogen peroxide and a reductive half where a primary amine is oxidized to an aldehyde. In one or more aspects, this reaction is reversible in the presence of ammonia, hydrogen peroxide, and an aldehyde. Examples of CAOs that may be suitable for use in the present disclosure include without limitation MdAO2 enzyme from apple (Malus domestica), which demonstrates a specific activity of 0.0239 U mg⁻¹ on EA; Rhodococcus opacus A01 and A02 which were shown to have activity on EA with A01 exhibiting a k_{cat} of 3.3 s⁻¹ and Kₘ of 2.05 mM; and a secreted CAO from Sycephalastrum racemosum strain M0945, SrAOX (GenBank AB828595), was demonstrated to have a high specific activity of 7.01 U mg⁻¹ on EA. SrAOX is not only expressible in E. coli, but can also be purified in functional form from this host, showing that TPQ and copper incorporation is achievable without further intervention. The affinity of SrAOX for EA is much higher than that of the Arthrobacter sp. EAO described below (Kₘ of mM compared with 15 mM).

In an aspect, the finishing catalyst is an ethanolamine oxidase (EAO). EAO is a homodimeric type I AMO similar to CAOs with a subunit mass of approximately 70 kDa. In an aspect, an EAO suitable for use in the present disclosure is sourced from Arthrobacter sp. (ArEAO) that was found to have a specific activity on ethanolamine of 9 U mg⁻¹. ArEAO can be heterologously expressed in E. coli.

In an aspect, the finishing catalyst is a glyoxalase. The major system for reactive α-carbonyl species (e.g., methylglyoxal (MG) and glyoxal (GO) detoxification in both prokaryotes and eukaryotes involves two enzymes, glyoxalase I (GLO1, EC 4.4.1.5) and glyoxalase II (GLO2, EC 3,1.2.6). GLO1 converts MG into S-D-lactoylglutathione (SLG) with glutathione (GSH) as a catalytic cofactor, and then GLO2 hydrolyzes SLG to D-lactate and GSH. Most recently, a GSH-independent glyoxalase system was identified in Escherichia coli, Caenorhabditis eiegans, mice and humans. In this system, glyoxalase III (GLY III) converts MG directly into D-lactate in a single step, with no cofactors. The enzyme instead relies on a conserved catalytic glu/asp-cys-tyr/his triad in its active site with the cysteine playing the pivotal role in catalysis. The in vitro activity of glyoxalase III is typically higher than the glyoxalase I/II system that generates D-lactate using a glutathione cofactor. In an aspect, the GLY III is sourced as the product of E. coli gene hchA, also known as Hsp31.

In an aspect, a GLYIII suitable for use in the present disclosure has any of SEQ ID NO: 91 through SEQ ID NO:112.

In an aspect, the finishing catalyst is a small molecule chemical catalyst such as an acid or base. Examples of acids or bases suitable for use as a finishing catalyst include without limitation hydrochloric acid, sulfuric acid, formic acid, sodium hydroxide and urea.

In an aspect, the biocatalysts suitable for use as an HVCOS of the type disclosed herein may further include one or more purified cofactors. Herein, a cofactor refers to non-protein chemical compound that modulates the biological activity of the biocatalyst. Many enzymes require cofactors to function properly. Nonlimiting examples of purified enzyme cofactors suitable for use in the present disclosure include thiamine pyrophosphate, NAD+, NADP+, pyridoxal phosphate, methyl cobalamin, cobalamine, biotin, Coenzyme A, tetrahydrofolic acid, menaquinone, ascorbic acid, flavin mononucleotide, flavin adenine dinucleotide, and Coenzyme F420. Such cofactors may be included in the biocatalyst preparation and/or be added at various points during the reaction. In some aspects, cofactors included with the biocatalyst preparation may be readily regenerated with oxygen and/or may remain stable throughout the lifetime of the enzyme(s).

As will be understood by one of ordinary skill in the art with the benefit of the present disclosure, reactions of the type disclosed herein (e.g., biocatalyst oxidation of ethylene glycol) may result in the production of byproducts (e.g., hydrogen peroxide) that can detrimentally impact other components of the reaction mixture. For example, hydrogen peroxide may degrade the biocatalyst resulting in a loss of catalytic activity. In such aspects, mitigation of the detrimental effects of hydrogen peroxide may be carried out such as by the introduction of a catalase (E.C. 1.11.1.61), the use of a hydrogen peroxide-resistant biocatalyst, or combinations thereof.

In an aspect, a biocatalyst of the type disclosed herein is a wild type enzyme, a functional fragment thereof, or a functional variant thereof. As used herein, “fragment” is meant to include any amino acid sequence shorter than the full-length biocatalyst (e.g., AOX), but where the fragment maintains a catalytic activity sufficient to meet some user or process goal. Fragments may include a single contiguous sequence identical to a portion of the biocatalyst sequence. Alternatively, the fragment may have or include several different shorter segments where each segment is identical in amino acid sequence to a different portion of the amino acid sequence of the biocatalyst but linked via amino acids differing in sequence from the biocatalyst. Herein, a “functional variant” of the biocatalyst refers to a polypeptide which has at one or more positions of an amino acid insertion, deletion, or substitution, either conservative or non-conservative, and wherein each of these types of changes may occur alone, or in combination with one or more of the others, one or more times in a given sequence but retains catalytic activity.

In the alternative or in combination with the aforementioned mutations, the biocatalyst may be mutated to improve the catalytic activity. Mutations may be carried out in order to enhance the protein or a homolog activity, increase the protein stability in the presence of glycolaldehyde and/or hydrogen peroxide, and increase protein yield.

Herein, reference has been made to “sources” of biocatalysts. It is to be understood this refers to the biomolecule as expressed by the named organism. It is contemplated the biocatalyst may be obtained from the organism or a version of said biocatalyst (wildtype or recombinant) provided as a suitable construct to an appropriate expression system.

In an aspect, any biocatalyst of the type disclosed herein may be cloned into an appropriate expression vector and used to transform cells of an expression system such as E. coli, Saccharomyces sp., Pichia sp., Aspergillus sp., Trichoderma sp., or Myceliophthora sp. A “vector” is a replicon, such as plasmid, phage, viral construct or cosmid, to which another DNA segment may be attached. Vectors are used to transduce and express a DNA segment in cells. As used herein, the terms “vector” and “construct” may include replicons such as plasmids, phage, viral constructs, cosmids, Bacterial Artificial Chromosomes (BACs), Yeast Artificial Chromosomes (YACs) Human Artificial Chromosomes (HACs) and the like into which one or more gene expression cassettes may be or are ligated. Herein, a cell has been “transformed” by an exogenous or heterologous nucleic acid or vector when such nucleic acid has been introduced inside the cell, for example, as a complex with transfection reagents or packaged in viral particles. The transforming DNA may or may not be integrated (covalently linked) into the genome of the cell.

In an aspect, the gene of a biocatalyst disclosed herein is provided as a recombinant sequence in a vector where the sequence is operatively linked to one or more control or regulatory sequences. “Operatively linked” expression control sequences refers to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and are used herein refer to polynucleotide sequences, which are necessary to affect the expression of coding sequences to which they are operatively linked. Expression control sequences are sequences that control the transcription, post-transcriptional events, and translation of nucleic acid sequences. Expression control sequences include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

In an aspect, an HVCOS is used in the production of formaldehyde. In such aspects, the input comprises methanol, the oxidation biocatalyst comprises an AOX, the oxidized intermediate comprises formaldehyde, the finishing catalyst comprises urea, and the output comprises a urea formaldehyde polymer. In such aspects, the oxidation biocatalyst, AOX is utilized in a bubble column reactor where oxygen is introduced under pressure to increase availability to the enzyme. The oxidation biocatalyst generates formaldehyde and hydrogen peroxide. The peroxide can be catalytically disproportionated into water and oxygen (e.g., using a catalase) to preserve enzyme function or hydrogen peroxide may be recovered as a coproduct. This is depicted in Reaction Scheme 3.

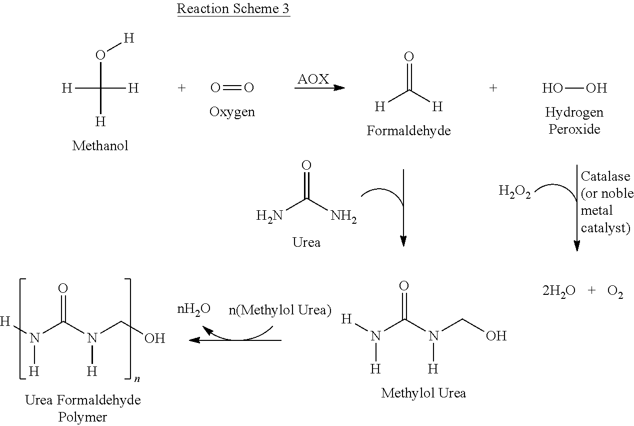

Because formaldehyde is a known crosslinker, it may be necessary to maintain low formaldehyde concentrations as it is generated through subsequent chemical reactions to protect the AOX, further enrichment and purification trains are not shown. It has been noted that AOX activity is negatively affected by aldehydes in two ways: 1) competitive inhibition by the aldehyde product with rapid equilibrium and 2) covalent interaction between the aldehyde and the enzyme. Short urea formaldehyde polymers precipitate from solution as a white solid until the condensation products reach a length at which the product remains soluble at room temperature. Upon further polymerization, the viscosity of the solution increases until an insoluble gel is irreversibly formed. These properties facilitate recovery of formaldehyde products from the reactor. The remaining solution containing a lower amount of formaldehyde and unreacted methanol can be recycled to the enzyme reactor. Other chemicals that may be useful for stabilizing produced formaldehyde include melamine, hydrazine hydrate, methyl-cellulose, guanamines, and bismelamines.

In an aspect, an HVCOS is used in the production of lactate. In such aspects, the input comprises glycerol, the oxidation biocatalyst comprises a GlyOX, the oxidized intermediate is L-glyceraldehyde, the finishing catalyst is an acid/base catalyst and the outputs are dihydroxyacetone, pyruvaldehyde (also known as methylglyoxal, or MG) and D-lactate. The reaction is depicted in Reaction Scheme 4.

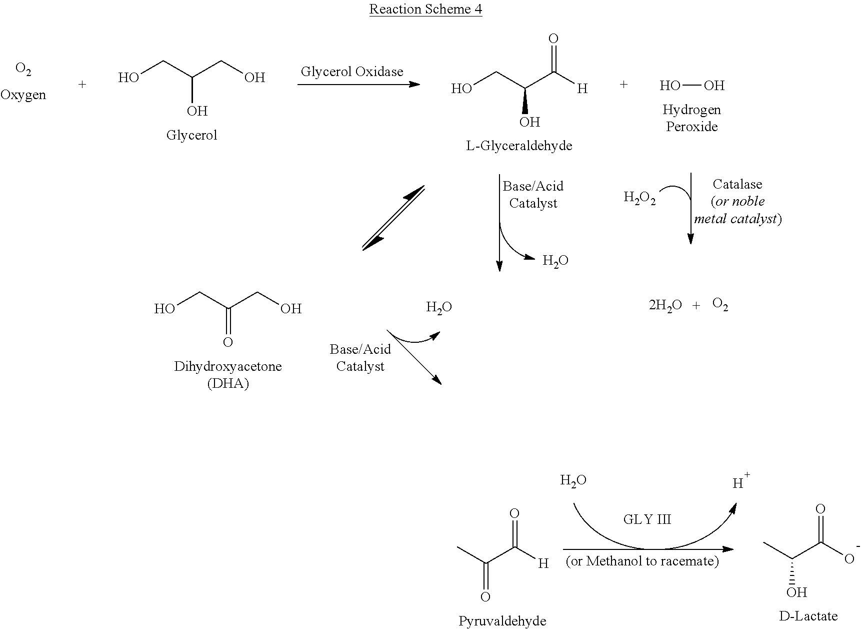

Referring to Reaction Scheme 4, GlyOX oxidizes glycerol to glyceraldehyde, generating hydrogen peroxide upon reduction of molecular oxygen. Hydrogen peroxide can be degraded with enzyme or retained. Glyceraldehyde, which exists in equilibrium with dihydroxyacetone, can be further dehydrated to methylglyoxal in the presence of an acid or base catalyst. Stereoselective conversion of MG to D-lactic acid then occurs in the presence of GLYIII. Generation of the lactic acid racemate may occur by treatment of the L-glyceraldehyde with enough base or acid to bypass the MG intermediate or by treating MG with methanol.

In an aspect, an HVCOS is used in the production of ethanolamine. In such aspects, the input comprises ethylene glycol, the oxidation biocatalyst comprises a CRO, a GAO, an AOX, a GlyOx or combinations thereof; the oxidized intermediate comprises a glycolaldehyde; the finishing catalyst comprises an AMO and the output comprises ethanolamine. The process is generally depicted in Reaction Scheme 5.

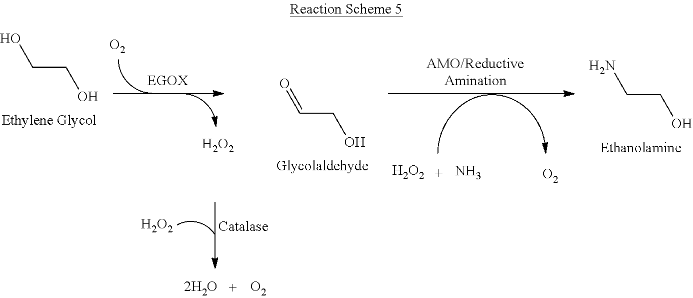

In an aspect, an HVCOS is used in the production of glycerol. In such aspects, the input comprises ethylene glycol, the oxidation biocatalyst comprises an EgOX, the oxidized intermediate comprises a glycolaldehyde; the finishing catalyst comprises a carboligating catalyst, a TMC or both; and the output comprises glycerol. The reaction is generally depicted in Reaction Scheme 6.

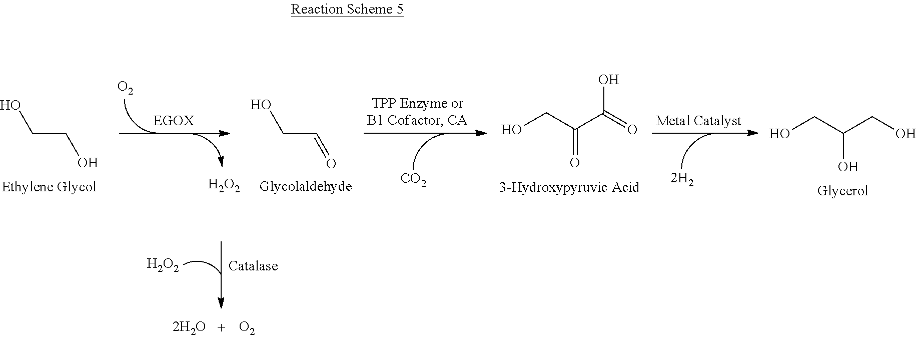

Referring to Reaction Scheme 6, the HVCOS production comprises conversion of ethylene glycol to glycolaldehyde. In an aspect, EgOx catalyzes the conversion of ethylene glycol to form the intermediate, glycolaldehyde, using molecular oxygen and generating hydrogen peroxide. Glycolaldehyde and carbon dioxide in the presence of the finishing catalyst, a carboligation catalyst is converted to 3-hydroxypyruvic acid that is subsequently reduced by a TMC to glycerol.

In an aspect, an HVCOS is used in the production of acetaldehyde. In such aspects, the input comprises ethanol; the oxidation biocatalyst comprises AOX and the output is acetaldehyde. The reaction is generally depicted in Reaction Scheme 6. In this aspect, AOX may be added either as a purified protein, or by the addition of whole cell Pichia Pastoris with AOX expressed.

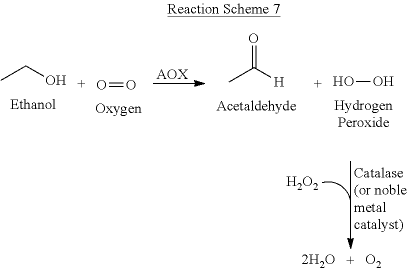

In an aspect, an HVCOS is used in the production of dihydroxyacetone (DHA). In such aspects, the input comprises glycerol; the oxidation biocatalyst comprises an AOX, a AldO, a CRO, a GlyOX or combinations thereof; the intermediate comprises glyceraldehyde the finishing catalyst comprise an isomerization catalyst and the output comprises DHA. This is depicted in Reaction Scheme 8.

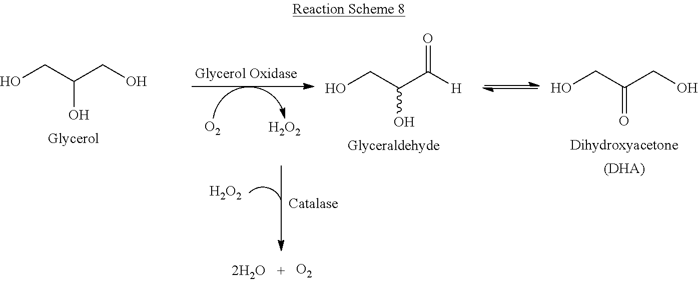

### Examples

The subject matter having been generally described, the following examples are given as particular aspects of the disclosure and are included to demonstrate the practice and advantages thereof, as well as aspects and features of the presently disclosed subject matter. It should be appreciated by those of skill in the art that the techniques disclosed in the examples that follow represent techniques discovered by the inventors to function well in the practice of the present subject matter, and thus can be considered to constitute preferred modes for its practice. However, those of skill in the art should, in light of the present disclosure, appreciate that many changes can be made in the specific aspects which are disclosed and still obtain a like or similar result without departing from the scope of the instant disclosure. It is understood that the examples are given by way of illustration and are not intended to limit the specification of the claims to follow in any manner.

A mutated Fusarium graminearum GAO (FgGAO) and a native GAO homolog from Colletotrichum spinosum (CsAlcOX) has both been shown to be capable of oxidizing ethylene glycol. The FgGAO sequence containing the M1 mutations and R330K, Q406T, and W290F, as well as the C383S mutation was able to oxidize ethylene glycol with a specific activity of about 18 U mL⁻¹ bacterial lysate and 3.9 U mg⁻¹ with purified protein as while CsAlcOX exhibited a specific activity for the oxidation of ethylene glycol of about 0.6 U mL⁻¹. Studies are underway to elucidate mutations in the FgGAO M-RQW-S scaffold to enhanced stability and activity.

### Example Protocol for Enzyme Testing

### Colorimetric Microtiter Plate Screening

Mutants of biocatalysts may be screened using a microtiter plate-base colorimetric assay that monitors the production of hydrogen peroxide. For example, the reagents o-dianisidine and 2,2′-azino-bis(3-ethylbenzothiazoline-6-sulphonic acid) (ABTS) may be used in conjunction with horseradish peroxidase to elicit a color change in the presence of hydrogen peroxide. Enzymes or combination of enzymes are diluted to total stock concentration of 1 mg/mL and then diluted further into a roughly 200 μL volume containing glucose substrate, the reporter molecule, buffer, and horseradish peroxidase. The dilution factor is chosen such that the change in color falls within the range of 0.01-0.06 absorbance units per minute as measured with a plate-based spectrophotometer. This rate of change can be used along with the dilution factor and extinction coefficient of the reporter molecule to calculate the specific activity of the enzyme(s) or plotted to select mutants with high activity for further characterization.

### Parr Bomb Scale Testing

Once suitable enzymes have been identified through the screening process, a pressurized Parr bomb system will be used to mimic the reactor conditions at scale in order to assess the efficacy of converting an input (e.g., ethylene glycol) to a HVC (e.g., ethanolamine) using the disclosed processes. In a final volume of about 50 mL, biocatalysts at amounts ranging from about 0.001 percent weight by volume (w/v %) to about 1 w/v % will be combined with approximately 20 w/v % input (e.g., ethylene glycol) and a suitable buffer at an initial pH in the range of from about 4 to about 7. Catalase may be added to provide a ratio of from about 1:1 to 1:20 biocatalyst to catalase ratio to prevent accumulation of hydrogen peroxide. The mixture will be loaded into the Parr bomb containing a stir bar. To improve mass transfer of oxygen into the solution, the vessel will be sparged with oxygen two times, then pressurized to 100 psig. The reactor will be held at constant temperature, typically about 20° C. but within the range of from about 10° C. to about 80° C. and the mixture allowed to react until the reaction is complete. During the reaction, the vessel may be depressurized to adjust the pH and obtain samples to assess conversion and product profile. Testing methods may include monitoring pH decrease as acids are produced, a colorimetric o-dianisidine assay to monitor formation of hydrogen peroxide, and HPLC for the detection of glycolaldehyde and EA.

Reactions of the type disclosed herein are carried out in a reactor system. Such reactor systems may utilize multiple reactors, but it could be a single continuous stirred tank slurry reactor (CSTR) or comprise a plurality of reactors (e.g., fixed bed reactors) of various sizes with or without interstage cooling and interstage caustic injection. Similarly, the enzymatic reactor could be a sparged bubble column or an air lift column or a falling film high pressure oxidation vessel. In an aspect, the inputs and oxidation biocatalyst are dosed into a bubble column reactor sparged with air to improve oxygen mass transfer. Typical operating ranges for the Enzyme Reactor is 20-60° C. at pressures between 1 and 15 bar. pH can be controlled by the addition of strong acids, bases, or buffers. Enzyme reactor effluent may be sent to a tangential flow filter (TFF) to preserve enzymes in the enzyme reactor as recycled retentate, with permeate flowing further down the process.

### Additional Disclosure

The following are non-limiting, specific aspects in accordance with the present disclosure:

A first aspect which is a system for the production of high value chemicals, comprising (a) an input selected from the group consisting of ethylene glycol, glycerol, ethanol methanol or a combination thereof; (b) an oxidation biocatalyst comprising an alcohol oxidase, a copper radical oxidase, a glycerol oxidase, an alditol oxidase or a combination thereof; (c) an oxidized intermediate; (d) a finishing catalyst comprising a supported metal catalyst, a carboligating catalyst, an amine oxidase, a glyoxalase, an acid catalyst, a base catalyst, an isomerization catalyst or a combination thereof; and (e) an output.

A second aspect which is the system of the first aspect wherein the alcohol oxidase has any of SEQ ID NO: 1 to SEQ ID NO: 71.

A third aspect which is the system of any of the first through second aspects wherein the carboligating catalyst has any of SEQ ID NO: 72 to SEQ ID NO: 86.

A fourth aspect which is the system of any of the first through third aspects wherein the carboligating catalyst has SEQ ID NO: 113.

A fifth aspect which is the system of any of the first through fourth aspects wherein the amine oxidase has any of SEQ ID NO: 87 to SEQ ID NO: 90.

A sixth aspect which is the system of any of the first through fifth aspects wherein the glyoxalase has any of SEQ ID NO: 91 to SEQ ID NO: 112.

A seventh aspect which is the system of any of the first through sixth aspects wherein the supported metal catalyst comprises a nanoparticle support.

An eighth aspect which is the system of any of the first through seventh aspects wherein the support of the supported metal catalyst comprises the support comprises carbon, silica, surface treated alumina, titania (TiO₂), zirconia (ZrO₂), a zeolite, montmorillonites, or a combination thereof.

A ninth aspect which is the system of any of the first through eighth aspects wherein the supported metal catalyst comprises a Group 8 metal, a 3d transition metal, an early transition metal, or combinations thereof.

A tenth aspect which is the system of any of the first through ninth aspects wherein the supported metal catalyst comprises gold, platinum or a combination thereof.

An eleventh aspect which is the system of any of the first through tenth aspects wherein the carboligating catalyst comprises pyruvate decarboxylase, formolase, the E1 component of α-ketoglutarate dehydrogenase complex from SucA, the KdcA gene product of Lactococcus lactis, a cofactor, or a combination thereof.

A twelfth aspect which is the system of any of the first through eleventh aspects wherein (i) the input comprises methanol; (ii) the oxidation biocatalyst comprises an alcohol oxidase; (iii) the intermediate comprises formaldehyde; (iv) the finishing catalyst comprises urea; and (v) the output comprises a urea formaldehyde polymer.

A thirteenth aspect which is the system of any of the first through twelfth aspects further comprising a catalase.

A fourteenth aspect which is the system of any of the first through thirteenth aspects wherein (i) the input comprises glycerol; (ii) the oxidation biocatalyst comprises a glycerol oxidase; (iii) the oxidized intermediate comprises L-glyceraldehyde; (iv) the finishing catalyst comprises an acid catalyst; and (v) the output comprises D-lactate.

A fifteenth aspect which is the system of any of the first through fourteenth aspects wherein (i) the input comprises ethylene glycol; (ii) the oxidation biocatalyst comprises a copper radical oxidase, a galactose oxidase, an alcohol oxidase, a glycerol oxidase or a combination thereof; (iii) the oxidized intermediate comprises a glycolaldehyde; (iv) the finishing catalyst comprises an amine monooxidase; and (v) the output comprises ethanolamine.

A sixteenth aspect which is the system of any of the first through fourteenth aspects wherein (i) the input comprises ethylene glycol; (ii) the oxidation biocatalyst comprises an ethylene glycol oxidase; (iii) the oxidized intermediate comprises a glycolaldehyde; (iv) the finishing catalyst comprises a carboligating catalyst, a supported metal catalyst or a combination thereof; and (v) the output comprises glycerol.

A seventeenth aspect which is the system of any of the first through fourteenth aspects wherein (i) the input comprises ethanol; (ii) the oxidation biocatalyst comprises an alcohol oxidase; (iii) the oxidized intermediate and (iv) the output comprises an acetaldehyde.

An eighteenth aspect which is the system of the seventeenth aspect further comprising a catalase.

A nineteenth aspect which is the system of any of the first through fourteenth aspects wherein (i) the input comprises glycerol; (ii) the oxidation biocatalyst comprises an alcohol oxidase, an alditol oxidase, a copper-radical oxidase, a glycerol oxidase or a combination thereof; (iii) the oxidized intermediate comprises glyceraldehyde; (iv) the finishing catalyst comprises an isomerization catalyst; and (v) the output comprises dihydroxyacetone.

A twentieth aspect which is the system of any of the first through nineteenth aspects wherein the output has a percentage purity of from about 60% to about 95%.

A twenty-first aspect which is a method for the production of high value chemicals, comprising:(a) contacting an input selected from the group consisting of ethylene glycol, glycerol, ethanol methanol or a combination thereof with an oxidation biocatalyst comprising an alcohol oxidase, a copper radical oxidase, a glycerol oxidase, an alditol oxidase or a combination thereof to form an oxidized intermediate; (b) contacting the oxidized intermediate with a finishing catalyst comprising a supported metal catalyst, a carboligating catalyst, an amine oxidase, a glyoxalase, an acid catalyst, a base catalyst, an isomerization catalyst or a combination thereof to form an outpyt.

A twenty-second aspect which is the method of the twenty-first aspect wherein the alcohol oxidase has any of SEQ ID NO: 1 to SEQ ID NO: 71.

A twenty-third aspect which is the method of any of the twenty-first through twenty-second aspects wherein the carboligating catalyst has any of SEQ ID NO: 72 to SEQ ID NO: 86.

A twenty-fourth aspect which is the method of any of the twenty-first through twenty-third aspects wherein the carboligating catalyst has SEQ ID NO: 113.

A twenty-fifth aspect which is the method of any of the twenty-first through twenty-fourth aspects wherein the amine oxidase has any of SEQ ID NO: 87 to SEQ ID NO: 90.

A twenty-sixth aspect which the method of any of the twenty-first through twenty-fifth aspects wherein the glyoxalase has any of SEQ ID NO: 91 to SEQ ID NO: 112.

A twenty-seventh aspect which is the method of any of the twenty-first through twenty-sixth aspects wherein the supported metal catalyst comprises a nanoparticle support.

A twenty-eighth aspect which is the method of any of the twenty-first through twenty-seventh aspects the carboligating catalyst comprises pyruvate decarboxylase, formolase, the E1 component of α-ketoglutarate dehydrogenase complex from SucA, the KdcA gene product of Lactococcus lactis, a cofactor, or a combination thereof.

A twenty-ninth aspect which is the method of any of the twenty-first through twenty-eight aspects wherein the output has a percentage purity of from about 60% to about 95%.

The subject matter having been shown and described, modifications thereof can be made by one skilled in the art without departing from the spirit and teachings of the subject matter. The aspects described herein are exemplary only and are not intended to be limiting. Many variations and modifications of the subject matter disclosed herein are possible and are within the scope of the disclosed subject matter. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an aspect of the present disclosure. Thus, the claims are a further description and are an addition to the aspects of the present invention. The discussion of a reference herein is not an admission that it is prior art to the presently disclosed subject matter, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A system for the production of high value chemicals, comprising:
(a) an input selected from the group consisting of ethylene glycol, glycerol, ethanol methanol and a combination thereof;
(b) an oxidation biocatalyst comprising an alcohol oxidase, a copper radical oxidase, a glycerol oxidase, an alditol oxidase or a combination thereof;
(c) an oxidized intermediate;
(d) a finishing catalyst comprising a supported metal catalyst, a carboligating catalyst, an amine oxidase, a glyoxalase, an acid catalyst, a base catalyst, an isomerization catalyst or a combination thereof; and
(e) an output.

2. The system of claim 1, wherein the alcohol oxidase has any of SEQ ID NO: 1 to SEQ ID NO: 71.

3. The system of claim 1, wherein the carboligating catalyst has any of SEQ ID NO: 72 to SEQ ID NO: 86.

4. The system of claim 1, wherein the carboligating catalyst has SEQ ID NO: 113.

5. The system of claim 1, wherein the amine oxidase has any of SEQ ID NO: 87 to SEQ ID NO: 90.

6. The system of claim 1, wherein the glyoxalase has any of SEQ ID NO: 91 to SEQ ID NO: 112.

7. The system of claim 1, wherein the supported metal catalyst comprises a nanoparticle support.

8. The system of claim 1, wherein the support of the supported metal catalyst comprises the support comprises carbon, silica, surface treated alumina, titania (TiO₂), zirconia (ZrO₂), a zeolite, montmorillonites, or a combination thereof.

9. The system of claim 1, wherein the supported metal catalyst comprises a Group 8 metal, a 3d transition metal, an early transition metal, or combinations thereof.

10. The system of claim 1, wherein the supported metal catalyst comprises gold, platinum or a combination thereof.

11. The system of claim 1, wherein the carboligating catalyst comprises pyruvate decarboxylase, formolase, the E1 component of α-ketoglutarate dehydrogenase complex from SucA, the KdcA gene product of Lactococcus lactis, a cofactor, or a combination thereof.

12. The system of claim 1, wherein (i) the input comprises methanol; (ii) the oxidation biocatalyst comprises an alcohol oxidase; (iii) the intermediate comprises formaldehyde; (iv) the finishing catalyst comprises urea; and (v) the output comprises a urea formaldehyde polymer.

13. The system of claim 1, further comprising a catalase.

14. The system of claim 1, wherein (i) the input comprises glycerol; (ii) the oxidation biocatalyst comprises a glycerol oxidase; (iii) the oxidized intermediate comprises L-glyceraldehyde; (iv) the finishing catalyst comprises an acid catalyst; and (v) the output comprises D-lactate.

15. The system of claim 1, wherein (i) the input comprises ethylene glycol; (ii) the oxidation biocatalyst comprises a copper radical oxidase, a galactose oxidase, an alcohol oxidase, a glycerol oxidase or a combination thereof; (iii) the oxidized intermediate comprises a glycolaldehyde; (iv) the finishing catalyst comprises an amine monooxidase; and (v) the output comprises ethanolamine.

16. The system of claim 1, wherein (i) the input comprises ethylene glycol; (ii) the oxidation biocatalyst comprises an ethylene glycol oxidase; (iii) the oxidized intermediate comprises a glycolaldehyde; (iv) the finishing catalyst comprises a carboligating catalyst, a supported metal catalyst or a combination thereof; and (v) the output comprises glycerol.

17. The system of claim 1, wherein (i) the input comprises ethanol; (ii) the oxidation biocatalyst comprises an alcohol oxidase; (iii) the oxidized intermediate and (iv) the output comprises an acetaldehyde.

18. The system of claim 17, further comprising a catalase.

19. The system of claim 1, wherein (i) the input comprises glycerol; (ii) the oxidation biocatalyst comprises an alcohol oxidase, an alditol oxidase, a copper-radical oxidase, a glycerol oxidase or a combination thereof; (iii) the oxidized intermediate comprises glyceraldehyde; (iv) the finishing catalyst comprises an isomerization catalyst; and (v) the output comprises dihydroxyacetone.

20. The system of claim 1, wherein the output has a percentage purity of from about 60% to about 95%.

21. A method for the production of high value chemicals, comprising:
(a) contacting an input selected from the group consisting of ethylene glycol, glycerol, ethanol methanol and a combination thereof with an oxidation biocatalyst comprising an alcohol oxidase, a copper radical oxidase, a glycerol oxidase, an alditol oxidase or a combination thereof to form an oxidized intermediate;
(b) contacting the oxidized intermediate with a finishing catalyst comprising a supported metal catalyst, a carboligating catalyst, an amine oxidase, a glyoxalase, an acid catalyst, a base catalyst, an isomerization catalyst or a combination thereof to form an output.

22. The method of claim 21, wherein the alcohol oxidase has any of SEQ ID NO: 1 to SEQ ID NO: 71.

23. The method of claim 21, wherein the carboligating catalyst has any of SEQ ID NO: 72 to SEQ ID NO: 86.

24. The method of claim 21, wherein the carboligating catalyst has SEQ ID NO: 113.

25. The method of claim 21, wherein the amine oxidase has any of SEQ ID NO: 87 to SEQ ID NO: 90.

26. The method of claim 21, wherein the glyoxalase has any of SEQ ID NO: 91 to SEQ ID NO: 112.

27. The method of claim 21, wherein the supported metal catalyst comprises a nanoparticle support.

28. The method of claim 21, wherein the carboligating catalyst comprises pyruvate decarboxylase, formolase, the E1 component of α-ketoglutarate dehydrogenase complex from SucA, the KdcA gene product of Lactococcus lactis, a cofactor, or a combination thereof.

29. The method of claim 21, wherein the output has a percentage purity of from about 60% to about 95%.
